(19) United States
(12) Patent Application Publication
(10) Pub. No.: US 2025/0002503 A1
(43) Pub. Date: Jan. 2, 2025

(54) OXADIAZASPIRO COMPOUNDS FOR USE IN THE TREATMENT OF MOTONEURON DEGENERATION OR IN NEUROPROTECTION

(71) Applicant: ESTEVE PHARMACEUTICALS, S.A., Barcelona (ES)

(72) Inventors: José-Miguel VELA-HERNÁNDEZ, Barcelona (ES); Manuel MERLOS-ROCA, BARCELONA (ES); Xavier NAVARRO-ACEBES, Barcelona (ES); Mireia HERRANDO-GRABULOSA, Centelles (ES)

(21) Appl. No.: 18/692,906

(22) PCT Filed: Sep. 16, 2022
(86) PCT No.: PCT/EP2022/075782

§ 371 Date: Mar. 18, 2024

(30) Foreign Application Priority Data
Sep. 20, 2021 (EP) EP21382845.2

(51) Int. Cl. C07D 498/10; A61K 31/5386; A61P 21/00; A61P 25/28

(52) U.S. Cl. CPC C07D 498/10; A61K 31/5386; A61P 21/00; A61P 25/28

(57) ABSTRACT

The present invention relates to compounds having pharmacological activity towards the sigma (#) receptor, and more particularly to oxadiazaspiro compounds having this pharmacological activity, for use in neuroprotection or in the treatment of motoneuron degeneration.

### FIELD OF THE INVENTION

The present invention relates to oxadiazaspiro compounds, or a pharmaceutical acceptable salt thereof, for use in neuroprotection or in the treatment of motoneuron degeneration.

### BACKGROUND

Motor neuron diseases (MND) represent a heterogeneous group of chronic sporadic and hereditary neurological disorders involving the upper or lower motor neurons (MNs), mainly represented by amyotrophic lateral sclerosis (ALS) in adults and spinal muscular atrophy (SMA) in children. There is no effective treatment available yet for most of these diseases; three drugs based on gene therapy are approved for SMA and only two drugs are approved for ALS, riluzole and edaravone, which slightly prolong the lifespan of the patients (Edaravone (MCI-186) ALS 19 Study Group, 2017; Ludolph and Jesse, 2009). This is in good part due to the multiple etiopathogenetic mechanisms, with a proposed complex interplay between excitotoxicity, neuroinflammation, oxidative stress, protein aggregation, mitochondrial dysfunction, and axonal transport defects, contributing to MN degeneration (Mancuso and Navarro, 2015).

Sigma-1 receptor (Sig-1R) is a protein enriched in the endoplasmic reticulum of MNs. Although, it has no direct downstream signaling, Sig-1R acts as a chaperone modulating plenty of essential cellular processes (Langa et al., 2003). Several mutations on the Sig-1R gene have been identified that lead to different types of MND, such as a severe, juvenile-onset form of ALS (ALS16) (Al-Saif et al., 2011), development of FTD-ALS (Luty et al., 2010), and some familial cases of distal hereditary motor neuropathies (dHMN) (Ververis et al., 2019). In addition, the administration of Sig-1R ligands was shown to exert neuroprotection in various experimental models of MN degeneration, including in vitro excitotoxicity (Guzmán-Lenis et al., 2009) and in vivo models of ALS (Mancuso et al., 2012b), of spinal root injury (Penas et al., 2011) and on the wobbler mouse which is a model of spontaneous MN degeneration (Peviani et al., 2014). Taken together, all these data illustrate the connection between Sig-1R and MN survival.

Accordingly, there is a need to find compounds exerting outstanding neuroprotection activity for the treatment of motoneuron degeneration.

Oxadiazaspiro compounds selectively binding to the sigma-1 receptor, surprisingly display strong neuroprotection activity in the treatment of neurodegeneration conditions.

Oxadiazaspiro compounds are such promising ligands. These compounds and their synthesis are disclosed and claimed in WO 2017084752.

### BRIEF DESCRIPTION OF THE FIGURES

FIG. 1. Chemical structure of compound A (compound 89 of the list) and compound B (compound 93 of the list) from the same chemical series and PRE-084.

FIG. 2. Compound A and compound B ligands prevent MN death under chronic excitotoxicity. A) Representative confocal images of spinal cord ventral horns labeled with SM132 antibody at 28 DIV for control, THA and THA plus compound A or compound B treated at four concentrations (0.03, 0.3, 3 and 30 μM). Scale bar 100 μm. B—C) Bar graphs showing the number of SMI-32 positive cells of each spinal cord hemislice.

FIG. 3. Plasma and brain concentration-time curves of compound A (A) and compound B (B) after single intraperitoneal administration of 10 mg/kg to mice.

FIG. 4. Administration of compound A and compound B increased MN survival and modulated glia activation after L4-L5 spinal roots injury. A) Plot of number of α-MNs in L4-L5 segments. B) Representative images corresponding to ventral horns of L4-L5 cord segments ipsilateral to rhizotomy in female mice with or without Sig-1R ligands treatment at 42 dpi. Scale bar 100 μm. C) Representative images of glial reactivity assessed by microglia (IBA1) and astrocytes (GFAP) immunolabeling in the ventral horn of control and rhizotomized mice with vehicle or Sig-1R treatment at 42 dpi. Scale bar 50 μm. D-E) Bar graph showing the integrated density of Iba-1 and GFAP immunolabeling in the ipsilateral ventral horn of spinal cord. F) Plot of the body weight of rhizotomized mice during the study.

FIG. 5. Effects of administration of compound A and compound B on disease progression of SOD1^{G93A} mice. A-B) Plots of the CMAP amplitude of plantar and tibialis anterior (TA) muscles. C) Rotarod performance in treated SOD1^{G93A} mice after treatment with Compound A and Compound B. D) Histological analyses of NMJ in the GM muscle of treated SOD1^{G93A} with Compound A and Compound B compared to vehicle group. E) Plot of the body weight of the different groups of mice during the follow-up. F) Representative confocal images of NMJ in the GM muscle of WT mice and SOD1^{G93A} male mice with or without Sig-1R ligands treatment at 16 weeks of age. Scale bar 100 μm.

FIG. 6. Neuroprotective effects of compound A and compound B administration on MNs and glial reactivity in SOD1^{G93A} mice at 16 weeks. A) Representative images of the ventral horns stained with cresyl violet of WT and SOD1^{G93A} male mice with or without Sig-1R ligands treatment. Scale bar 100 μm. B) Histological analyses of MNs in mice treated with compound A, compound B and PRE-084. C-D) Bar graph showing the integrated density of Iba-1 and GFAP immunolabeling in the ventral horn of spinal cord. D) Representative images of glial reactivity assessed by IBA and GFAP to label microglia and astrocytes in the ventral horn. Scale bar 50 μm.

### SUMMARY OF THE INVENTION

The invention is directed in a main aspect to a compound of general Formula (I),

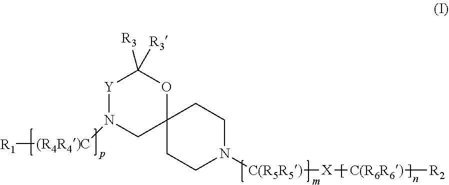

wherein R₁, R₂, R₃, R_{3′}, R₄, R_{4′}, R₅, R_{5′}, R₆, R_{6′}, X, Y, m, n and p are as defined below in the detailed description, for use in neuroprotection or in the treatment of motoneuron degeneration.

In another aspect, the present invention relates to neuroprotection or to a method of treatment of motoneuron degeneration comprising administering to a subject in need thereof a therapeutically effective amount of a compound of Formula (I).

In a further aspect, the present invention relates to a pharmaceutical composition which comprises a compound of Formula (I) or a pharmaceutically acceptable salt thereof, and a pharmaceutically acceptable carrier, adjuvant or vehicle for use in neuroprotection or in the treatment of motoneuron degeneration.

### DETAILED DESCRIPTION OF THE INVENTION

The present invention discloses compounds with affinity to sigma receptors for use in neuroprotection or in the treatment of motoneuron degeneration.

In a particular aspect, the present invention is directed to compounds of general Formula (I):

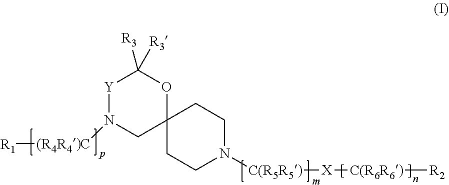

wherein
p is 0 or 1 or 2;
m is 1, 2 or 3;
n is 0, 1 or 2;
Y is —CH₂— or —C(O)—;
X is a bond, —C(RₓR_{x′})—, —C(O)— or —O—;
   wherein Rₓ is selected from halogen, substituted or unsubstituted C₁₋₆ alkyl, substituted or unsubstituted C₂₋₆ alkenyl substituted or unsubstituted C₂₋₆ alkynyl, and —OR₈;
   R_{x′} is selected from hydrogen, halogen or substituted or unsubstituted C₁₋₆ alkyl, substituted or unsubstituted C₂₋₆ alkenyl and substituted or unsubstituted C₂₋₆ alkynyl;
   R₈ is selected from hydrogen, substituted or unsubstituted C₁₋₆ alkyl, substituted or unsubstituted C₂₋₆ alkenyl and substituted or unsubstituted C₂₋₆ alkynyl;
R₁ is selected from substituted or unsubstituted C₁₋₆ alkyl, substituted or unsubstituted C₂₋₆ alkenyl, substituted or unsubstituted C₂₋₆ alkynyl and substituted or unsubstituted cycloalkyl;
R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl;
R₃ and R_{3′}, are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R₃ and R_{3′}, taken together with the connecting C-atom may form a substituted or unsubstituted cycloalkyl;
R₄ and R_{4′}, are independently selected from hydrogen, substituted or unsubstituted C₁₋₉ alkyl, substituted or unsubstituted C₂₋₉ alkenyl, substituted or unsubstituted C₂₋₉ alkynyl, —CHOR₉ and —C(O)OR₉;
   wherein R₉ is selected from hydrogen, substituted or unsubstituted C₁₋₉ alkyl, substituted or unsubstituted C₂₋₉ alkenyl and substituted or unsubstituted C₂₋₉ alkynyl;
R₅ and R_{5′} are independently selected from hydrogen, substituted or unsubstituted C₁₋₆ alkyl, substituted or unsubstituted C₂₋₆ alkenyl, substituted or unsubstituted C₂₋₆ alkynyl, —CHOR₇ and —C(O)OR₇;
   wherein R₇ is selected from hydrogen, substituted or unsubstituted C₁₋₆ alkyl, substituted or unsubstituted C₂₋₆ alkenyl and substituted or unsubstituted C₂₋₆ alkynyl;
alternatively, R₅ and R_{5′} taken together with the connecting C-atom may form a substituted or unsubstituted cycloalkyl or a substituted or unsubstituted non-aromatic heterocyclyl;
R₆ and R_{6′}, are independently selected from hydrogen, substituted or unsubstituted C₂₋₆ alkyl, substituted or unsubstituted C₂₋₆ alkenyl, substituted or unsubstituted C₂₋₆ alkynyl, —OR₁₀, —CHOR₁₀ and —C(O)OR₁₀;
   wherein R₁₀ is selected from hydrogen, substituted or unsubstituted C₁₋₆ alkyl, substituted or unsubstituted C₂₋₆ alkenyl and substituted or unsubstituted C₂₋₆ alkynyl;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof, for use in neuroprotection or in the treatment of motoneuron degeneration.

These compounds for use according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In another embodiment, these compounds for use according to the invention are optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof.

In a further embodiment the compound of general Formula (I) is a compound of general Formula (I′)

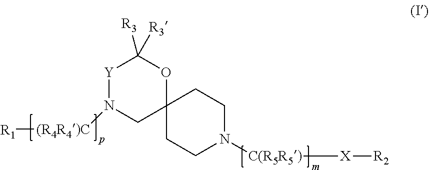

wherein, R₁, R₂, R₃, R₃, R₄, R_{4′}, R₅, R_{5′}, X, Y, m and p are as defined in the description, for use in neuroprotection or in the treatment of motoneuron degeneration.

In a further embodiment the compound of general Formula (I) is a compound of general Formula (I^{a}′)

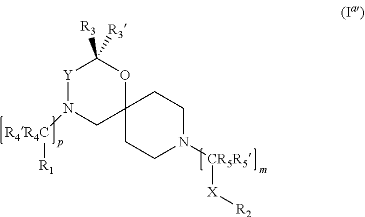

wherein, R₁, R₂, R₃, R₃, R₄, R_{4′}, R₅, R_{5′}, X, Y, m and p are as defined in the description, for use in neuroprotection or in the treatment of motoneuron degeneration.

In a further embodiment the compound of general Formula (I) is a compound of general Formula (I^{b}′)

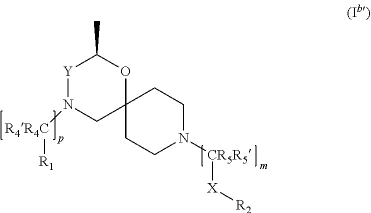

wherein, R₁, R₂, R₄, R_{4′}, R₅, R_{5′}, X, Y, m and p are as defined in the description, for use in neuroprotection or in the treatment of motoneuron degeneration.

In a further embodiment the compound of general Formula (I) is a compound of general Formula (I^{c}′)

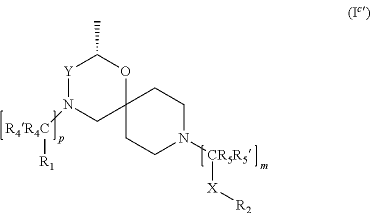

wherein, R₁, R₂, R₄, R_{4′}, R₅, R_{5′}, X, Y, m and p are as defined in the description, for use in neuroprotection or in the treatment of motoneuron degeneration.

In a further embodiment the compound of general Formula (I) is a compound of general Formula (I²′)

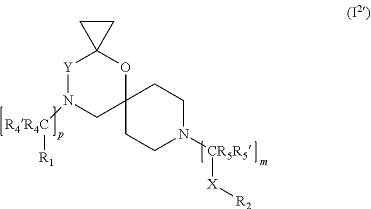

wherein R₁, R₂, R₄, R_{4′}, R₅, R_{5′}, X, Y, m and p are as defined in the description, for use in neuroprotection or in the treatment of motoneuron degeneration.

In a further embodiment, compounds of general Formula (I) are compounds of general Formula (I³′)

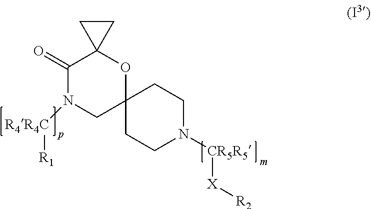

wherein R₁, R₂, R₄, R_{4′}, R₅, R_{5′}, X, m and p are as defined in the description, for use in neuroprotection or in the treatment of motoneuron degeneration.

In a further embodiment, compounds of general Formula (I) are compounds of general Formula (I^{4′})

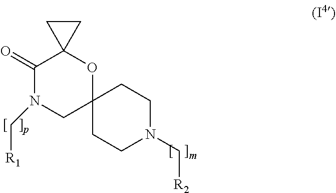

wherein R₁, R₂, m and p are as defined in the description, for use in neuroprotection or in the treatment of motoneuron degeneration.

In a further embodiment, compounds of general Formula (I) are compounds of general Formula (I^{5′})

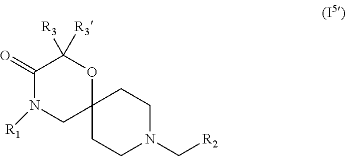

wherein R₁, R₂, R₃ and R_{3′} are as defined in the description, for use in neuroprotection or in the treatment of motoneuron degeneration.

In a further embodiment the compound of general Formula (I) is a compound of general Formula (I^{6′})

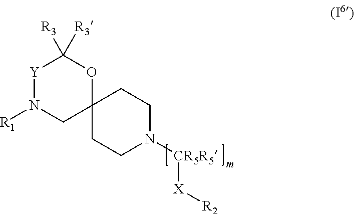

wherein, R₁, R₂, R₃, R_{3′}, R₅, R_{5′}, X, Y, and m are as defined in the description, for use in neuroprotection or in the treatment of motoneuron degeneration.

For clarity purposes, reference is also made to the following statements below in the definitions of substitutions on alkyl etc. or aryl etc. that “wherein when different radicals R₁ to R_{14″″} and Rₓ, R_{x′} are present simultaneously in Formula I they may be identical or different”. This statement is reflected in the below general Formula (I^{7′}) being derived from and falling into general Formula (I).

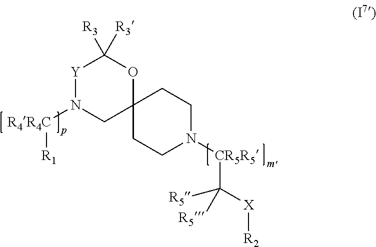

wherein R₁, R₂, R₃, R_{3′}, R₄, R_{4′}, R₅, R_{5′}, X, Y and p are as defined in the description. In addition, m′ (being 0 or 1), R_{F} and R₅ are added. As said above, this statement is thus reflected in that R_{5″} and R_{5′″} are or could be different from R₅ and R_{5′} or not and—accordingly—m′ being 0 or 1 is naturally resulting from m (in general Formulas (I) to (I^{6′}) being 1 or 2).

The same would be applicable mutatis mutandis for general Formulas like general Formula (I) as well as the other general Formulas (I′) to (I^{6′}) above, as well as to all the intermediates of synthesis.

For clarity purposes, all groups and definitions described in the description and referring to compounds of general Formula (I), also apply to compounds of general Formula (I′), (I^{a}′), (I^{b}′), (I^{c}′), (I²′), (I³′), (I⁴′), (I⁵′), or (I⁶˜) and also (I⁷′), as well as to all the intermediates of synthesis, when those groups are present in the mentioned general Markush formulae, since compounds of general Formula (I′), (I^{a}′), (I^{b}′), (I^{c}′), (I²′), (I³′), (I⁴′), (I⁵′), (I⁶′) or (I′) are included in the general Formula (I).

In the context of this invention, alkyl is understood as meaning saturated, linear or branched hydrocarbons, which may be unsubstituted or substituted once or several times. It encompasses e.g. —CH₃ and —CH₂—CH₃. In these radicals, C₁₋₂-alkyl represents C1- or C2-alkyl, C₁₋₃-alkyl represents C1-, C2- or C3-alkyl, C₁₋₄-alkyl represents C1-, C2-, C3- or C4-alkyl, C₁₋₅-alkyl represents C1-, C2-, C3-, C4-, or C5-alkyl, C₁₋₆-alkyl represents C1-, C2-, C3-, C4-, C5- or C6-alkyl, C₁₋₇-alkyl represents C1-, C2-, C3-, C4-, C5-, C6- or C7-alkyl, C₁₋₃-alkyl represents C1-, C2-, C3-, C4-, C5-, C6-, C7- or C8-alkyl, C₁₋₁₀-alkyl represents C1-, C2-, C3-, C4-, C5-, C6-, C7-, C8-, C9- or C10-alkyl and C₁₋₁₈-alkyl represents C1-, C2-, C3-, C4-, C5-, C6-, C7-, C8-, C9-, C10-, C11-, C12-, C13-, C14-, C15-, C16-, C17- or C18-alkyl. The alkyl radicals are preferably methyl, ethyl, propyl, methylethyl, butyl, 1-methylpropyl, 2-methylpropyl, 1,1-dimethylethyl, pentyl, 1,1-dimethylpropyl, 1,2-dimethylpropyl, 2,2-dimethylpropyl, hexyl, 1-methylpentyl, if substituted also CHF₂, CF₃ or CH₂OH etc. Preferably alkyl is understood in the context of this invention as C₁₋₈alkyl like methyl, ethyl, propyl, butyl, pentyl, hexyl, heptyl, or octyl; preferably is C₁₋₆alkyl like methyl, ethyl, propyl, butyl, pentyl, or hexyl; more preferably is C₁₋₄alkyl like methyl, ethyl, propyl or butyl.

Alkenyl is understood as meaning unsaturated, linear or branched hydrocarbons, which may be unsubstituted or substituted once or several times. It encompasses groups like e.g. —CH═CH—CH₃. The alkenyl radicals are preferably vinyl (ethenyl), allyl (2-propenyl). Preferably in the context of this invention alkenyl is C₂₋₁₀-alkenyl or C₂₋₈-alkenyl like ethylene, propylene, butylene, pentylene, hexylene, heptylene or octylene; or is C₂₋₆ alkenyl like ethylene, propylene, butylene, pentylene, or hexylene; or is C₂₋₄-alkenyl, like ethylene, propylene, or butylenes.

Alkynyl is understood as meaning unsaturated, linear or branched hydrocarbons, which may be unsubstituted or substituted once or several times. It encompasses groups like e.g. —C≡C—CH₃ (1-propinyl). Preferably alkynyl in the context of this invention is C₂₋₁₀-alkynyl or C₂₋₈-alkynyl like ethyne, propyne, butyene, pentyne, hexyne, heptyne, or octyne; or is C₂₋₆-alkynyl like ethyne, propyne, butyene, pentyne, or hexyne; or is C₂₋₄-alkynyl like ethyne, propyne, butyene, pentyne, or hexyne.

In connection with alkyl (also in alkylaryl, alkylheterocyclyl or alkylcycloalkyl), alkenyl, alkynyl and O-alkyl—unless defined otherwise—the term substituted in the context of this invention is understood as meaning replacement of at least one hydrogen radical on a carbon atom by halogen (F, Cl, Br, I), —NR_{c}R_{c″″}, —SR_{c}, —S(O)R_{c}, —S(O)₂R_{c}, —OR_{c}, —C(O)OR_{c}, —CN, —C(O)NR_{c}R_{c′}, haloalkyl, haloalkoxy or —OC₁₋₆alkyl, being R_{c} represented by R₁₁, R₁₂, R₁₃, (being R_{c′} represented by R_{11′}, R_{12′}, R_{13′}; being R_{c″} represented by R_{11″}, R_{12″}, R_{13″}; being R_{c′″}, represented by R_{11′″}, R_{12′″}, R_{13′″}, being R_{c″″} represented by R_{11″″}, R_{12″″}, R_{13″″}) wherein R₁ to R_{14″″} and Rₓ, R_{x′} and Rₙ are as defined in the description, and wherein when different radicals R₁ to R_{14″″} and Rₓ, R_{x′} and Rₙ are present simultaneously in Formula I they may be identical or different.

Most preferably in connection with alkyl (also in alkylaryl, alkylheterocyclyl or alkylcycloalkyl), alkenyl, alkynyl or O-alkyl, substituted is understood in the context of this invention that any alkyl (also in alkylaryl, alkylheterocyclyl or alkylcycloalkyl), alkenyl, alkynyl or O-alkyl which is substituted is substituted with one or more of halogen (F, Cl, Br, I), —OR_{c}, —CN, —NR_{c}R_{c″″}, haloalkyl, haloalkoxy or —OC₁₋₆alkyl, being R_{c} represented by R₁₁, R₁₂, R₁₃, (being R_{c′} represented by R_{11′}, R_{12′}, R_{13′}; being R_{c″} represented by R_{11″}, R_{12″}, R_{13″}; being R_{c′″}, represented by R_{11′″}, R_{12′″}, R_{13′″}, being R_{c″″} represented by R_{11″″}, R_{12″″}, R_{13″″}), wherein R₁ to R_{14″″} and Rₓ, R_{x′} and Rₙ are as defined in the description, and wherein when different radicals R₁ to R₁₄ and Rₓ, R_{x′} and Rₙ are present simultaneously in Formula I, they may be identical or different.

More than one replacement on the same molecule and also on the same carbon atom is possible with the same or different substituents. This includes for example 3 hydrogens being replaced on the same C atom, as in the case of CF₃, or at different places of the same molecule, as in the case of e.g. —CH(OH)—CH═CH—CHCl₂.

In the context of this invention haloalkyl is understood as meaning an alkyl being substituted once or several times by a halogen (selected from F, Cl, Br, I). It encompasses e.g. —CH₂Cl, —CH₂F, —CHCl₂, —CHF₂, —CCl₃, —CF₃ and —CH₂—CHCl₂. Preferably haloalkyl is understood in the context of this invention as halogen-substituted C₁₋₄-alkyl representing halogen substituted C1-, C2-, C3- or C4-alkyl. The halogen-substituted alkyl radicals are thus preferably methyl, ethyl, propyl, and butyl. Preferred examples include —CH₂C1, —CH₂F, —CHCl₂, —CHF₂, and —CF₃.

In the context of this invention haloalkoxy is understood as meaning an —O-alkyl being substituted once or several times by a halogen (selected from F, Cl, Br, 1). It encompasses e.g. —OCH₂Cl, —OCH₂F, —OCHCl₂, —OCHF₂, —OCCl₃, —OCF₃ and —OCH₂—CHCl₂. Preferably haloalkyl is understood in the context of this invention as halogen-substituted —OC₁₋₄-alkyl representing halogen substituted C1-, C2-, C3- or C4-alkoxy. The halogen-substituted alkyl radicals are thus preferably O-methyl, O-ethyl, O-propyl, and O-butyl. Preferred examples include —OCH₂Cl, —OCH₂F, —OCHCl₂, —OCHF₂, and —OCF₃.

In the context of this invention cycloalkyl is understood as meaning saturated and unsaturated (but not aromatic) cyclic hydrocarbons (without a heteroatom in the ring), which can be unsubstituted or once or several times substituted. Furthermore, C₃₋₄-cycloalkyl represents C3- or C4-cycloalkyl, C₃₋₅-cycloalkyl represents C3-, C4- or C5-cycloalkyl, C₃₋₆-cycloalkyl represents C3-, C4-, C5- or C6-cycloalkyl, C₃₋₇-cycloalkyl represents C3-, C4-, C5-, C6- or C7-cycloalkyl, C₃₋₈-cycloalkyl represents C3-, C4-, C5-C6-, C7- or C8-cycloalkyl, C₄₋₅-cycloalkyl represents C4- or C5-cycloalkyl, C₄₋₆-cycloalkyl represents C4-, C5- or C6-cycloalkyl, C₄₋₇-cycloalkyl represents C4-, C5-, C6- or C7-cycloalkyl, C₅₋₆-cycloalkyl represents C5- or C6-cycloalkyl and C₅₋₇-cycloalkyl represents C5-, C6- or C7-cycloalkyl. Examples are cyclopropyl, 2-methylcyclopropyl, cyclopropylmethyl, cyclobutyl, cyclopentyl, cyclopentylmethyl, cyclohexyl, cycloheptyl, cyclooctyl, and also adamantly. Preferably in the context of this invention cycloalkyl is C₃₋₆cycloalkyl like cyclopropyl, cyclobutyl, cyclopentyl, cyclohexyl, cycloheptyl, or cyclooctyl; or is C₃₋₇cycloalkyl like cyclopropyl, cyclobutyl, cyclopentyl, cyclohexyl, or cycloheptyl; or is C₃₋₆cycloalkyl like cyclopropyl, cyclobutyl, cyclopentyl or cyclohexyl, especially cyclopentyl or cyclohexyl.

Aryl is understood as meaning 5 to 18 membered mono or polycyclic ring systems with at least one aromatic ring but without heteroatoms even in only one of the rings. Examples are phenyl, naphthyl, fluoranthenyl, fluorenyl, tetralinyl or indanyl, 9H-fluorenyl or anthracenyl radicals, which can be unsubstituted or once or several times substituted. Most preferably aryl is understood in the context of this invention as phenyl, naphtyl or anthracenyl, preferably is phenyl.

A heterocyclyl radical or group (also called heterocyclyl hereinafter) is understood as meaning 5 to 18 membered mono or polycyclic heterocyclic ring systems, with at least one saturated or unsaturated ring which contains one or more heteroatoms from the group consisting of nitrogen, oxygen and/or sulfur in the ring. A heterocyclic group can also be substituted once or several times.

Examples include non-aromatic heterocyclyls such as tetrahydropyrane, oxazepane, morpholine, piperidine, pyrrolidine as well as heteroaryls such as furan, benzofuran, thiophene, benzothiophene, pyrrole, pyridine, pyrimidine, pyrazine, quinoline, isoquinoline, phthalazine, thiazole, benzothiazole, indole, benzotriazole, carbazole and quinazoline.

Subgroups inside the heterocyclyls as understood herein include heteroaryls and non-aromatic heterocyclyls.
the heteroaryl (being equivalent to heteroaromatic radicals or aromatic heterocyclyls) is an aromatic 5 to 18 membered mono or polycyclic heterocyclic ring system of one or more rings of which at least one aromatic ring contains one or more heteroatoms from the group consisting of nitrogen, oxygen and/or sulfur in the ring; preferably is an aromatic 5 to 18 membered mono or polycyclic heterocyclic ring system of one or two rings of which at least one aromatic ring contains one or more heteroatoms from the group consisting of nitrogen, oxygen and/or sulfur in the ring, more preferably is selected from furan, benzofuran, thiophene, benzothiophene, pyrrole, pyridine, pyrimidine, pyrazine, quinoline, isoquinoline, phthalazine, benzothiazole, indole, benzotriazole, carbazole, quinazoline, thiazole, imidazole, pyrazole, oxazole, thiophene and benzimidazole;
the non-aromatic heterocyclyl is a 5 to 18 membered mono or polycyclic heterocyclic ring system of one or more rings of which at least one ring—with this (or these) ring(s) then not being aromatic—contains one or more heteroatoms from the group consisting of nitrogen, oxygen and/or sulfur in the ring; preferably is a 5 to 18 membered mono or polycyclic heterocyclic ring system of one or two rings of which one or both rings—with this one or two rings then not being aromatic—contain/s one or more heteroatoms from the group consisting of nitrogen, oxygen and/or sulfur in the ring, more preferably is selected from oxazepam, pyrrolidine, piperidine, piperazine, tetrahydropyran, morpholine, indoline, oxopyrrolidine, benzodioxane, oxetane, especially is benzodioxane, morpholine, tetrahydropyran, piperidine, oxopyrrolidine, oxetane and pyrrolidine.

Preferably, in the context of this invention heterocyclyl is defined as a 5 to 18 membered mono or polycyclic heterocyclic ring system of one or more saturated or unsaturated rings of which at least one ring contains one or more heteroatoms from the group consisting of nitrogen, oxygen and/or sulfur in the ring. Preferably it is a 5 to 18 membered mono or polycyclic heterocyclic ring system of one or two saturated or unsaturated rings of which at least one ring contains one or more heteroatoms from the group consisting of nitrogen, oxygen and/or sulfur in the ring.

Preferred examples of heterocyclyls include oxetane, oxazepan, pyrrolidine, imidazole, oxadiazole, tetrazole, pyridine, pyrimidine, piperidine, piperazine, benzofuran, benzimidazole, indazole, benzodiazole, thiazole, benzothiazole, tetrahydropyrane, morpholine, indoline, furan, triazole, isoxazole, pyrazole, thiophene, benzothiophene, pyrrole, pyrazine, pyrrolo[2,3b]pyridine, quinoline, isoquinoline, phthalazine, benzo-1,2,5-thiadiazole, indole, benzotriazole, benzoxazole oxopyrrolidine, pyrimidine, benzodioxolane, benzodioxane, carbazole and quinazoline, especially is pyridine, pyrazine, indazole, benzodioxane, thiazole, benzothiazole, morpholine, tetrahydropyrane, pyrazole, imidazole, piperidine, thiophene, indole, benzimidazole, pyrrolo[2,3b]pyridine, benzoxazole, oxopyrrolidine, pyrimidine, oxazepane, oxetane and pyrrolidine.

In the context of this invention oxopyrrolidine is understood as meaning pyrrolidin-2-one.

In connection with aromatic heterocyclyls (heteroaryls), non-aromatic heterocyclyls, aryls and cycloalkyls, when a ring system falls within two or more of the above cycle definitions simultaneously, then the ring system is defined first as an aromatic heterocyclyl (heteroaryl) if at least one aromatic ring contains a heteroatom. If no aromatic ring contains a heteroatom, then the ring system is defined as a non-aromatic heterocyclyl if at least one non-aromatic ring contains a heteroatom. If no non-aromatic ring contains a heteroatom, then the ring system is defined as an aryl if it contains at least one aryl cycle. If no aryl is present, then the ring system is defined as a cycloalkyl if at least one non-aromatic cyclic hydrocarbon is present.

In the context of this invention alkylaryl is understood as meaning an aryl group (see above) being connected to another atom through a C₁₋₆-alkyl (see above) which may be branched or linear and is unsubstituted or substituted once or several times.

Preferably alkylaryl is understood as meaning an aryl group (see above) being connected to another atom through 1 to 4 (—CH₂—) groups. Most preferably alkylaryl is benzyl (i.e. —CH₂-phenyl).

In the context of this invention alkylheterocyclyl is understood as meaning an heterocyclyl group being connected to another atom through a C₁₋₆-alkyl (see above) which may be branched or linear and is unsubstituted or substituted once or several times. Preferably alkylheterocyclyl is understood as meaning an heterocyclyl group (see above) being connected to another atom through 1 to 4 (—CH₂—) groups. Most preferably alkylheterocyclyl is —CH₂-pyridine.

In the context of this invention alkylcycloalkyl is understood as meaning an cycloalkyl group being connected to another atom through a C₁₋₆-alkyl (see above) which may be branched or linear and is unsubstituted or substituted once or several times. Preferably alkylcycloalkyl is understood as meaning a cycloalkyl group (see above) being connected to another atom through 1 to 4 (—CH₂—) groups. Most preferably alkylcycloalkyl is —CH₂-cyclopropyl.

Preferably, the aryl is a monocyclic aryl. More preferably the aryl is a 5, 6 or 7 membered monocyclic aryl. Even more preferably the aryl is a 5 or 6 membered monocyclic aryl.

Preferably, the heteroaryl is a monocyclic heteroaryl. More preferably the heteroaryl is a 5, 6 or 7 membered monocyclic heteroaryl. Even more preferably the heteroaryl is a 5 or 6 membered monocyclic heteroaryl.

Preferably, the non-aromatic heterocyclyl is a monocyclic non-aromatic heterocyclyl. More preferably the non-aromatic heterocyclyl is a 4, 5, 6 or 7 membered monocyclic non-aromatic heterocyclyl. Even more preferably the non-aromatic heterocyclyl is a 5 or 6 membered monocyclic non-aromatic heterocyclyl.

Preferably, the cycloalkyl is a monocyclic cycloalkyl. More preferably the cycloalkyl is a 3, 4, 5, 6, 7 or 8 membered monocyclic cycloalkyl. Even more preferably the cycloalkyl is a 3, 4, 5 or 6 membered monocyclic cycloalkyl.

In connection with aryl (including alkyl-aryl), cycloalkyl (including alkyl-cycloalkyl), or heterocyclyl (including alkyl-heterocyclyl), substituted is understood—unless defined otherwise—as meaning substitution of the ring-system of the aryl or alkyl-aryl, cycloalkyl or alkyl-cycloalkyl; heterocyclyl or alkyl-heterocyclyl with one or more of halogen (F, Cl, Br, I), —R_{c}, —OR_{c}, —CN, —NO₂, —NR_{c}R_{c′″}, —C(O)OR_{c}, NR_{c}C(O)R_{c′}, —C(O)NR_{c}R_{c″}, —NR_{c}S(O)₂R_{c′}, ═O, —OCH₂CH₂OH, —NR_{c}C(O)NR_{c′}R_{c′}, —S(O)₂NR_{c}R_{c″}, —NR_{c}S(O)₂NR_{c′}R_{c″}, haloalkyl, haloalkoxy, —SR_{c}, —S(O)R_{c}, —S(O)₂R_{c} or C(CH₃)OR_{c}; NR_{c}R_{c″″}, with R_{c}, R_{c′}, R_{c″} and R_{c′″} independently being either H or a saturated or unsaturated, linear or branched, substituted or unsubstituted C₁₋₆-alkyl; a saturated or unsaturated, linear or branched, substituted or unsubstituted C₁₋₆-alkyl; a saturated or unsaturated, linear or branched, substituted or unsubstituted —O—C₁₋₆-alkyl (alkoxy); a saturated or unsaturated, linear or branched, substituted or unsubstituted —S—C₁₋₆-alkyl; a saturated or unsaturated, linear or branched, substituted or unsubstituted —C(O)—C₁₋₆-alkyl-group; a saturated or unsaturated, linear or branched, substituted or unsubstituted —C(O)—O—C₁₋₆alkyl-group; a substituted or unsubstituted aryl or alkyl-aryl; a substituted or unsubstituted cycloalkyl or alkyl-cycloalkyl; a substituted or unsubstituted heterocyclyl or alkyl-heterocyclyl, being R_{c} one of R₁₁, R₁₂ or R₁₄, (being R_{c′} one of R_{11′}, R_{12′} or R_{14′}; being R_{c″} one of R_{11″}, R_{12″} or R_{14″}; being R_{c′″} one of R_{11′″}, R_{12′″} or R_{14′″}; being R_{c″″} one of R_{11″″}, R_{12″″} or R_{14″″}), wherein R₁ to R_{14″″} and Rₓ, R_{x′} and Rₙ are as defined in the description, and wherein when different radicals R₁ to R_{14″″} and Rₓ, R_{x′} and Rₙ are present simultaneously in Formula I they may be identical or different.

Most preferably in connection with aryl (including alkyl-aryl), cycloalkyl (including alkyl-cycloalkyl), or heterocyclyl (including alkyl-heterocyclyl), substituted is understood in the context of this invention that any aryl, cycloalkyl and heterocyclyl which is substituted is substituted (also in an alyklaryl, alkylcycloalkyl or alkylheterocyclyl) with one or more of halogen (F, Cl, Br, I), —R_{c}, —OR_{c}, —CN, —NO₂, —NR_{c}R_{c′″}, NR_{c}C(O)R_{c′}, —NR_{c}S(O)₂R_{c′}, ═O, haloalkyl, haloalkoxy, or C(CH₃)OR_{c}; —OC₁₋₄alkyl being unsubstituted or substituted with one or more of OR_{c} or halogen (F, Cl, I, Br), —CN, or —C₁₋₄alkyl being unsubstituted or substituted with one or more of OR_{c} or halogen (F, Cl, I, Br), being R_{c} one of R₁₁, R₁₂ or R₁₄, (being R_{c′} one of R_{11′}, R_{12′} or R_{14′}; being R_{c″} one of R_{11″}, R_{12″} or R_{14″}; being R_{c′″} one of R_{11′″}, R_{12′″}, or R_{14′″}; being R_{c″″} one of R_{11″″}, R_{12″″} or R_{14″″}), wherein R₁ to R_{14″″} and Rₓ, R_{x′} and Rₙ are as defined in the description, and wherein when different radicals R₁ to R_{14″″} and Rₓ, R_{x′} and Rₙ are present simultaneously in Formula I they may be identical or different.

Additionally to the above-mentioned substitutions, in connection with cycloalkyl (including alkyl-cycloalkyl), or heterocycly (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with

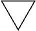

### or ═O.

In connection with cycloalkyl (including alkyl-cycloalkyl), or heterocycly (including alkylheterocyclyl) namely non-aromatic heterocyclyl (including non-aromatic alkyl-heterocyclyl), substituted is also understood—unless defined otherwise—as meaning substitution of the ring-system of the cycloalkyl or alkyl-cycloalkyl; non-aromatic heterocyclyl or non aromatic alkyl-heterocyclyl with

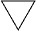

(leading to a spiro structure) or with ═O.

A ring system is a system consisting of at least one ring of connected atoms but including also systems in which two or more rings of connected atoms are joined with “joined” meaning that the respective rings are sharing one (like a spiro structure), two or more atoms being a member or members of both joined rings.

The term “leaving group” means a molecular fragment that departs with a pair of electrons in heterolytic bond cleavage. Leaving groups can be anions or neutral molecules. Common anionic leaving groups are halides such as Cl—, Br—, and I—, and sulfonate esters, such as tosylate (TsO—) or mesylate.

The term “salt” is to be understood as meaning any form of the active compound used according to the invention in which it assumes an ionic form or is charged and is coupled with a counter-ion (a cation or anion) or is in solution. By this are also to be understood complexes of the active compound with other molecules and ions, in particular complexes via ionic interactions.

The term “physiologically acceptable salt” means in the context of this invention any salt that is physiologically tolerated (most of the time meaning not being toxic-especially not caused by the counter-ion) if used appropriately for a treatment especially if used on or applied to humans and/or mammals.

These physiologically acceptable salts can be formed with cations or bases and in the context of this invention is understood as meaning salts of at least one of the compounds used according to the invention—usually a (deprotonated) acid—as an anion with at least one, preferably inorganic, cation which is physiologically tolerated—especially if used on humans and/or mammals. The salts of the alkali metals and alkaline earth metals are particularly preferred, and also those with NH₄, but in particular (mono)- or (di)sodium, (mono)- or (di)potassium, magnesium or calcium salts.

Physiologically acceptable salts can also be formed with anions or acids and in the context of this invention is understood as meaning salts of at least one of the compounds used according to the invention as the cation with at least one anion which are physiologically tolerated—especially if used on humans and/or mammals. By this is understood in particular, in the context of this invention, the salt formed with a physiologically tolerated acid, that is to say salts of the particular active compound with inorganic or organic acids which are physiologically tolerated—especially if used on humans and/or mammals. Examples of physiologically tolerated salts of particular acids are salts of: hydrochloric acid, hydrobromic acid, sulfuric acid, methanesulfonic acid, formic acid, acetic acid, oxalic acid, succinic acid, malic acid, tartaric acid, mandelic acid, fumaric acid, lactic acid or citric acid.

In a further embodiment the compound of general Formula (I) for use according to the invention is a compound wherein p is 0 or 1.

In another preferred embodiment of the compound of general Formula (I) for use according to the invention is a compound wherein X is a bond.

In another preferred embodiment of the compound of general Formula (I) for use according to the invention is a compound wherein X is —C(RₓR_{x′})—. In another preferred embodiment of the compound of general Formula (I) for use according to the invention is a compound wherein X is —O—.

In another preferred embodiment of the compound of general Formula (I) for use according to the invention is a compound wherein X is —C(O)—.

In a further embodiment the compound of general Formula (I) for use according to the invention is a compound wherein Y is —CH₂—.

In a further embodiment the compound of general Formula (I) for use according to the invention is a compound wherein Y is —C(O)—.

In a further embodiment the compound of general Formula (I) for use according to the invention is a compound wherein R₁ is selected from substituted or unsubstituted C₁₋₆ alkyl, substituted or unsubstituted C₂₋₆ alkenyl and substituted or unsubstituted C₂₋₆ alkynyl.

In a further embodiment the compound of general Formula (I) for use according to the invention is a compound wherein R₁ is substituted or unsubstituted C₁₋₆ alkyl.

In a further embodiment the compound of general Formula (I) for use according to the invention is a compound wherein R₁ is substituted or unsubstituted cycloalkyl.

In a further embodiment the compound of general Formula (I) for use according to the invention is a compound wherein R₁ is selected from substituted or unsubstituted C₁₋₆ alkyl and substituted or unsubstituted cycloalkyl.

In a further embodiment the compound of general Formula (I) for use according to the invention is a compound wherein R₁ is selected from unsubstituted C₁₋₆ alkyl and unsubstituted cycloalkyl.

In a further embodiment the compound of general Formula (I) for use according to the invention is a compound wherein R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted aryl and substituted or unsubstituted heterocyclyl.

In a further embodiment the compound of general Formula (I) for use according to the invention is a compound wherein R₂ is selected from hydrogen, substituted or unsubstituted C₁₋₆ alkyl, substituted or unsubstituted cycloalkyl, substituted or unsubstituted aryl and substituted or unsubstituted heterocyclyl.

In a further embodiment the compound of general Formula (I) for use according to the invention is a compound wherein R₂ is selected from hydrogen, substituted or unsubstituted C₁₋₆ alkyl, substituted or unsubstituted aryl and substituted or unsubstituted heterocyclyl.

In a further embodiment the compound of general Formula (I) for use according to the invention is a compound wherein R₂ is selected from substituted or unsubstituted aryl and substituted or unsubstituted heterocyclyl.

In a further embodiment the compound of general Formula (I) for use according to the invention is a compound wherein R₂ is substituted or unsubstituted aryl.

In another preferred embodiment of the compound of general Formula (I) for use according to the invention is a compound wherein R₃ and R_{3′} are independently selected from hydrogen and substituted or unsubstituted C₁₋₆ alkyl.

In another preferred embodiment of the compound of general Formula (I) for use according to the invention is a compound wherein R₃ and R₃, are independently selected from hydrogen and unsubstituted C₁₋₆ alkyl.

In another preferred embodiment of the compound of general Formula (I) for use according to the invention is a compound wherein R₃ is substituted or unsubstituted C₁₋₆ alkyl while R_{3′} is hydrogen.

In another preferred embodiment of the compound of general Formula (I) for use according to the invention is a compound wherein R₃ is unsubstituted C₁₋₆ alkyl while R₃ is hydrogen.

In another preferred embodiment of the compound of general Formula (I) for use according to the invention is a compound wherein R₄ and R_{4′} are independently selected from hydrogen, substituted or unsubstituted C₁₋₉ alkyl, substituted or unsubstituted C₂₋₉ alkenyl and substituted or unsubstituted C₂₋₉ alkynyl.

In another preferred embodiment of the compound of general Formula (I) for use according to the invention is a compound wherein R₄ and R_{4′} are independently selected from hydrogen and substituted or unsubstituted C₁₋₉alkyl.

In another preferred embodiment of the compound of general Formula (I) for use according to the invention is a compound wherein R₅ and R_{5′} are independently selected from hydrogen, substituted or unsubstituted C₁₋₆ alkyl, substituted or unsubstituted C₂₋₆ alkenyl and substituted or unsubstituted C₂₋₆ alkynyl.

In another preferred embodiment of the compound of general Formula (I) for use according to the invention is a compound wherein R₅ and R_{5′} are independently selected from hydrogen and substituted or unsubstituted C₁₋₆ alkyl.

In another preferred embodiment of the compound of general Formula (I) for use according to the invention is a compound wherein R₅ and R_{5′} taken together with the connecting C-atom form a substituted or unsubstituted cycloalkyl or a substituted or unsubstituted non-aromatic heterocyclyl, m is 1, X is a bond, n is 0 and R₂ is hydrogen.

In another preferred embodiment of the compound of general Formula (I) for use according to the invention is a compound wherein R₅ and R_{5′} taken together with the connecting C-atom form a substituted or unsubstituted cycloalkyl or a substituted or unsubstituted saturated heterocyclyl, m is 1, X is a bond, n is 0 and R₂ is hydrogen.

In another preferred embodiment of the compound of general Formula (I) for use according to the invention is a compound wherein R₆ and R_{6′} are independently selected from hydrogen, substituted or unsubstituted C₁₋₆ alkyl, substituted or unsubstituted C₂₋₆ alkenyl and substituted or unsubstituted C₂₋₆ alkynyl.

In another preferred embodiment of the compound of general Formula (I) for use according to the invention is a compound wherein R₆ and R_{6′} are independently selected from hydrogen and substituted or unsubstituted C₁₋₆ alkyl.

In another preferred embodiment of the compound of general Formula (I) for use according to the invention is a compound wherein R₇ is selected from hydrogen and substituted or unsubstituted C₁₋₆ alkyl.

In another preferred embodiment of the compound of general Formula (I) for use according to the invention is a compound wherein R₈ is selected from hydrogen and substituted or unsubstituted C₁₋₆ alkyl.

In another preferred embodiment of the compound of general Formula (I) for use according to the invention is a compound wherein R₉ is selected from hydrogen and substituted or unsubstituted C₁₋₉ alkyl.

In another preferred embodiment of the compound of general Formula (I) for use according to the invention is a compound wherein R₁₀ is selected from hydrogen and substituted or unsubstituted C₁₋₆ alkyl.

In another preferred embodiment of the compound of general Formula (I) for use according to the invention is a compound wherein R₁₁, R_{11′}, and R_{11″} are independently selected from hydrogen and unsubstituted C₁₋₆ alkyl.

In another preferred embodiment of the compound of general Formula (I) for use according to the invention is a compound wherein R_{11′″} is selected from hydrogen and unsubstituted C₁₋₆ alkyl.

In another preferred embodiment of the compound of general Formula (I) for use according to the invention is a compound wherein R₁₂, R_{12′} and R_{12′″} are independently selected from hydrogen and unsubstituted C₁₋₆ alkyl; In another preferred embodiment of the compound of general Formula (I) for use according to the invention is a compound wherein R_{12′″} is selected from hydrogen and unsubstituted C₁₋₆ alkyl.

In another preferred embodiment of the compound of general Formula (I) for use according to the invention is a compound wherein R₁₃ is selected from hydrogen and unsubstituted C₁₋₆ alkyl; In another preferred embodiment of the compound of general Formula (I) for use according to the invention is a compound wherein R_{13′″} is selected from hydrogen and unsubstituted C₁₋₆ alkyl.

In another preferred embodiment of the compound of general Formula (I) for use according to the invention is a compound wherein R₁₄, R_{14′} and R_{14″} are independently selected from hydrogen, unsubstituted C₁₋₆ alkyl, unsubstituted aryl, unsubstituted cycloalkyl and unsubstituted heterocyclyl; In another preferred embodiment of the compound of general Formula (I) for use according to the invention is a compound wherein R_{14′″} is selected from hydrogen and unsubstituted C₁₋₆ alkyl;

In another preferred embodiment of the compound of general Formula (I) for use according to the invention is a compound wherein Rₓ is selected from halogen, substituted or unsubstituted C₁₋₆ alkyl and —OR₈;

In another preferred embodiment of the compound of general Formula (I) for use according to the invention is a compound wherein Re is selected from hydrogen, halogen or substituted or unsubstituted C₁₋₆ alkyl;

In a further preferred embodiment the compound of general Formula (I) is a compound of general Formula (I^{5′})

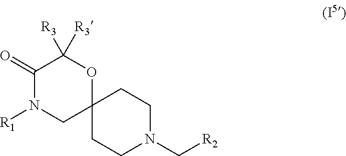

wherein,
R₁ is selected from unsubstituted C₁₋₆ alkyl and unsubstituted cycloalkyl;
R₂ is selected from substituted or unsubstituted aryl and substituted or unsubstituted heterocyclyl,
   wherein said aryl or heterocyclyl in R₂, if substituted, is substituted with one or more substituent/s selected from halogen, —OR₁₂, —CN and haloalkoxy;
   wherein R₁₂ is unsubstituted C₁₋₆ alkyl;
R₃ is unsubstituted C₁₋₆ alkyl;
R₃′ is hydrogen;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof, for use in neuroprotection or in the treatment of motoneuron degeneration.

In another preferred embodiment of the compound of general Formula (I) for use according to the invention is a compound, wherein
n is 0, 1 or 2; preferably, n is 0;

In another preferred embodiment of the compound of general Formula (I) for use according to the invention is a compound, wherein
m is 1, 2 or 3; preferably m is 1 or 2;

In another preferred embodiment of the compound of general Formula (I) for use according to the invention is a compound, wherein
p is 0 or 1 or 2;

In another preferred embodiment of the compound of general Formula (I) for use according to the invention is a compound, wherein
p is 0 or 1;

In another preferred embodiment of the compound of general Formula (I) for use according to the invention is a compound, wherein
X is a bond, —C(RₓR_{x′})—, —C(O)— or —O—; preferably, X is a bond or —O—;

In another preferred embodiment of the compound of general Formula (I) for use according to the invention is a compound, wherein
Y is —CH₂— or —C(O)—;

In another preferred embodiment of the compound of general Formula (I) for use according to the invention is a compound, wherein
X is a bond, —C(RₓR_{x′})—, —C(O)— or —O—; preferably X is a bond or —O— and/or
m is 1, 2 or 3; preferably m is 1 or 2; and/or
n is 0, 1 or 2; preferably n is 0; and/or
p is 0 or 1; preferably p is 0.

In a preferred embodiment
R₁ is a substituted or unsubstituted group selected from methyl, ethyl, propyl, isopropyl, isobutyl, sec-butyl, isopentyl, neo-pentyl and cyclopropyl.

In a preferred embodiment
R₁ is an unsubstituted group selected from methyl, ethyl, propyl, isopropyl, isobutyl, sec-butyl, isopentyl, neo-pentyl and cyclopropyl.

In a preferred embodiment
R₁ is a substituted or unsubstituted cyclopropyl, preferably unsubstituted cyclopropyl.

In a preferred embodiment
R₂ is hydrogen or a substituted or unsubstituted group selected from methyl, ethyl, propyl, isopropyl, isobutyl, tert-butyl, neo-pentyl, cyclopropyl, phenyl and pyridine; more preferably hydrogen or an unsubstituted group selected from methyl, ethyl, propyl, isopropyl, isobutyl, tert-butyl, neo-pentyl, cyclopropyl, or a substituted or unsubstituted group selected from phenyl and pyridine.

In a preferred embodiment
R₂ is a substituted or unsubstituted group selected from phenyl and pyridine.

In a preferred embodiment
R₂ is substituted or unsubstituted phenyl.

In a preferred embodiment
R₃ and R_{3′} taken together with the connecting C-atom may form a substituted or unsubstituted cyclopropyl: preferably unsubstituted cyclopropyl.

In a preferred embodiment
R₃ is hydrogen or substituted or unsubstituted methyl, preferably R_{3′} is hydrogen or unsubstituted methyl.

In a preferred embodiment
R_{3′} is hydrogen or substituted or unsubstituted methyl, preferably R_{3′} is hydrogen or unsubstituted methyl.

In a preferred embodiment
R_{3′} is hydrogen while R₃ is substituted or unsubstituted methyl, preferably R_{3′} is hydrogen while R₃ is unsubstituted methyl.

In a preferred embodiment
R₃ is hydrogen while R_{3′} is substituted or unsubstituted methyl, preferably R₃ is hydrogen while R_{3′} is unsubstituted methyl.

In a preferred embodiment
R₃ and R_{3′} are both substituted or unsubstituted methyl, preferably R₃ and R_{3′} are both unsubstituted methyl.

In a preferred embodiment
R₃ and R_{3′} are both hydrogen.

In a preferred embodiment
R₄ is hydrogen or substituted or unsubstituted methyl, preferably R₄ is hydrogen or unsubstituted methyl.

In a preferred embodiment
R_{4′} is hydrogen.

In a preferred embodiment
R_{4′} is hydrogen while R₄ is substituted or unsubstituted methyl, preferably R_{4′} is hydrogen while R₄ is unsubstituted methyl.

In a preferred embodiment
R₄ and R_{4′} are both hydrogen.

In a preferred embodiment
R₅ and R_{5′} are both hydrogen.

In a preferred embodiment
R₅ and R_{5′} taken together with the connecting C-atom form a substituted or unsubstituted group selected from cyclobutyl, cyclopentyl, cyclohexyl or tetrahydropyrane, preferably an unsubstituted group selected from cyclobutyl, cyclopentyl, cyclohexyl or tetrahydropyrane.

In a preferred embodiment
R₆ and R_{6′} are both hydrogen.

In a preferred embodiment
X is a bond.

In a preferred embodiment
X is —O—.

In a preferred embodiment
Y is —CH₂—;

In a preferred embodiment
Y is-C(O)—;

In another preferred embodiment
n is 0.

In another preferred embodiment
n is 1.

In another preferred embodiment
n is 2.

In another preferred embodiment
m is 1 or 2;

In another preferred embodiment
p is 0, 1 or 2.

In another preferred embodiment
p is 0 or 1.

In another preferred embodiment
p is 0.

In an particular embodiment
the halogen is fluorine or chlorine.

In an particular embodiment
the haloalkoxy is —OCF₃.

In an particular embodiment
the haloalkyl is —CF₃.

In another preferred embodiment
p is 0;
m is 1;
n is 0;
Y is —C(O)—;
X is a bond;
R₁ is selected from unsubstituted C₁₋₆ alkyl and unsubstituted cycloalkyl;
R₂ is selected from substituted or unsubstituted aryl and substituted or unsubstituted heterocyclyl,
   wherein said aryl or heterocyclyl in R₂, if substituted, is substituted with one or more substituent/s selected from halogen, —OR₁₂, —CN and haloalkoxy;
   wherein R₁₂ is unsubstituted C₁₋₆ alkyl;
R₃ is unsubstituted C₁₋₆ alkyl;
R_{3′} is selected from hydrogen and unsubstituted C₁₋₆ alkyl;
alternatively, R₃ and R_{3′} taken together with the connecting C-atom may form an unsubstituted cycloalkyl;
R₅ and R_{5′} are both hydrogen.

In another preferred embodiment
p is 0;
m is 1;
n is 0;
Y is —C(O)—;
X is a bond;
R₁ is unsubstituted C₁₋₆ alkyl;
R₂ is substituted or unsubstituted aryl;
wherein said aryl in R₂, if substituted, is substituted with one or more substituent/s selected from halogen and —CN;
R₃ is unsubstituted C₁₋₆ alkyl;
R_{3′} is hydrogen;
alternatively, R₃ and R₃, taken together with the connecting C-atom may form an unsubstituted cycloalkyl;
R₅ and R_{5′} are both hydrogen.

In a preferred further embodiment, the compounds of the general Formula (I) for use according to the invention are selected from

| NUMBER | STRUCTURE | CHEMICAL NAME |
|---|---|---|
| 1 | 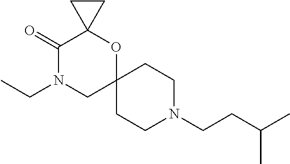 [SMILES: CCN1CC2(CCN(CCC(C)C)CC2)OC2(CC2)C1=O] | 12-ethyl-8-isopentyl-4-oxa-8,12- diazadispiro[2.1.5.3]tridecan-13-one |
| 2 | 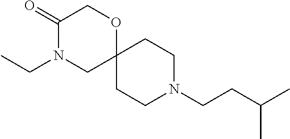 [SMILES: CCN1CC2(CCN(CCC(C)C)CC2)OCC1=O] | 4-ethyl-9-isopentyl-1-oxa-4,9- diazaspiro[5.5]undecan-3-one |
| 3 | 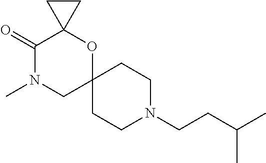 [SMILES: CC(C)CCN1CCC2(CC1)CN(C)C(=O)C1(CC1)O2] | 8-isopentyl-12-methyl-4-oxa-8,12- diazadispiro[2.1.5.3]tridecan-13-one |
| 4 | 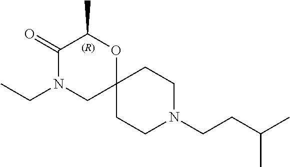 | (R)-4-ethyl-9-isopentyl-2-methyl-1-oxa-4,9- diazaspiro[5.5]undecan-3-one |
| 5 | 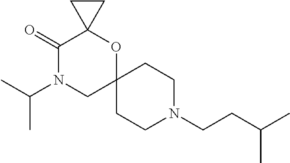 [SMILES: CC(C)CCN1CCC2(CC1)CN(C(C)C)C(=O)C1(CC1)O2] | 8-isopentyl-12-isopropyl-4-oxa-8,12- diazadispiro[2.1.5.3]tridecan-13-one |
| 6 | 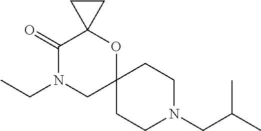 [SMILES: CCN1CC2(CCN(CC(C)C)CC2)OC2(CC2)C1=O] | 12-ethyl-8-isobutyl-4-oxa-8,12- diazadispiro[2.1.5.3]tridecan-13-one |
| 7 | 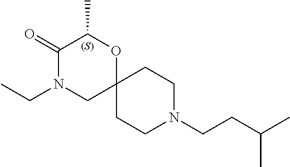 | (S)-4-ethyl-9-isopentyl-2-methyl-1-oxa-4,9- diazaspiro[5.5]undecan-3-one |
| 8 | 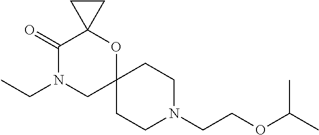 [SMILES: CCN1CC2(CCN(CCOC(C)C)CC2)OC2(CC2)C1=O] | 12-ethyl-8-(2-isopropoxyethyl)-4-oxa-8,12- diazadispiro[2.1.5.3]tridecan-13-one |
| 9 | 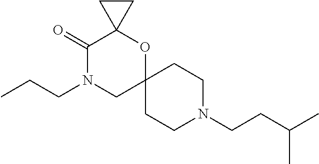 [SMILES: CCCN1CC2(CCN(CCC(C)C)CC2)OC2(CC2)C1=O] | 8-isopentyl-12-propyl-4-oxa-8,12- diazadispiro[2.1.5.3]tridecan-13-one |
| 10 | 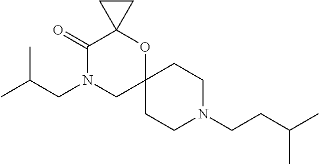 [SMILES: CC(C)CCN1CCC2(CC1)CN(CC(C)C)C(=O)C1(CC1)O2] | 12-isobutyl-8-isopentyl-4-oxa-8,12- diazadispiro[2.1.5.3]tridecan-13-one |
| 11 | 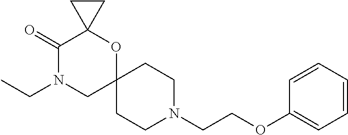 [SMILES: CCN1CC2(CCN(CCOc3ccccc3)CC2)OC2(CC2)C1=O] | 12-ethyl-8-(2-phenoxyethyl)-4-oxa-8,12- diazadispiro[2.1.5.3]tridecan-13-one |
| 12 | 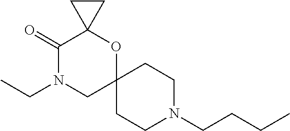 [SMILES: CCCCN1CCC2(CC1)CN(CC)C(=O)C1(CC1)O2] | 8-butyl-12-ethyl-4-oxa-8,12- diazadispiro[2.1.5.3]tridecan-13-one |
| 13 | 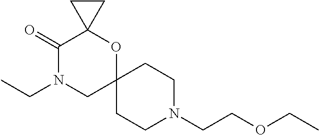 [SMILES: CCOCCN1CCC2(CC1)CN(CC)C(=O)C1(CC1)O2] | 8-(2-ethoxyethyl)-12-ethyl-4-oxa-8,12- diazadispiro[2.1.5.3]tridecan-13-one |
| 14 | 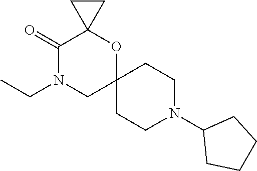 [SMILES: CCN1CC2(CCN(C3CCCC3)CC2)OC2(CC2)C1=O] | 8-cyclopentyl-12-ethyl-4-oxa-8,12- diazadispiro[2.1.5.3]tridecan-13-one |
| 15 | 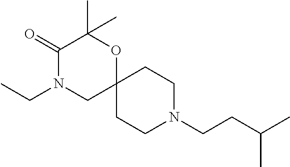 [SMILES: CCN1CC2(CCN(CCC(C)C)CC2)OC(C)(C)C1=O] | 4-ethyl-9-isopentyl-2,2-dimethyl-1-oxa-4,9- diazaspiro[5.5]undecan-3-one |
| 16 | 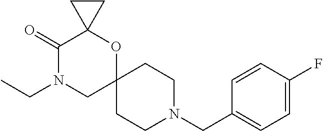 [SMILES: CCN1CC2(CCN(Cc3ccc(F)cc3)CC2)OC2(CC2)C1=O] | 12-ethyl-8-(4-fluorobenzyl)-4-oxa-8,12- diazadispiro[2.1.5.3]tridecan-13-one |
| 17 | 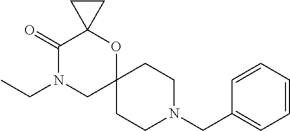 [SMILES: CCN1CC2(CCN(Cc3ccccc3)CC2)OC2(CC2)C1=O] | 8-benzyl-12-ethyl-4-oxa-8,12- diazadispiro[2.1.5.3]tridecan-13-one |
| 18 | 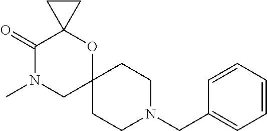 [SMILES: CN1CC2(CCN(Cc3ccccc3)CC2)OC2(CC2)C1=O] | 8-benzyl-12-methyl-4-oxa-8,12- diazadispiro[2.1.5.3]tridecan-13-one |
| 19 | 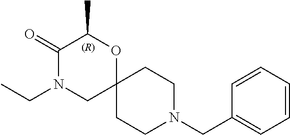 | (R)-9-benzyl-4-ethyl-2-methyl-1-oxa-4,9- diazaspiro[5.5]undecan-3-one |
| 20 | 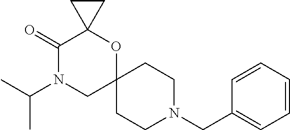 [SMILES: CC(C)N1CC2(CCN(Cc3ccccc3)CC2)OC2(CC2)C1=O] | 8-benzyl-12-isopropyl-4-oxa-8,12- diazadispiro[2.1.5.3]tridecan-13-one |
| 21 | 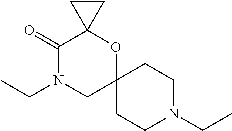 [SMILES: CCN1CCC2(CC1)CN(CC)C(=O)C1(CC1)O2] | 8,12-diethyl-4-oxa-8,12- diazadispiro[2.1.5.3]tridecan-13-one |
| 22 | 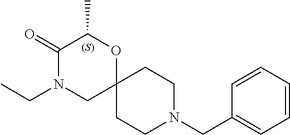 | (S)-9-benzyl-4-ethyl-2-methyl-1-oxa-4,9- diazaspiro[5.5]undecan-3-one |
| 23 | 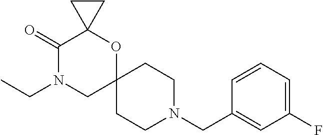 [SMILES: CCN1CC2(CCN(Cc3cccc(F)c3)CC2)OC2(CC2)C1=O] | 12-ethyl-8-(3-fluorobenzyl)-4-oxa-8,12- diazadispiro[2.1.5.3]tridecan-13-one |
| 24 | 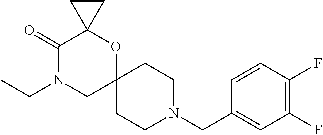 [SMILES: CCN1CC2(CCN(Cc3ccc(F)c(F)c3)CC2)OC2(CC2)C1=O] | 8-(3,4-difluorobenzyl)-12-ethyl-4-oxa-8,12- diazadispiro[2.1.5.3]tridecan-13-one |
| 25 | 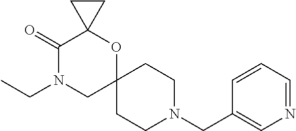 [SMILES: CCN1CC2(CCN(Cc3cccnc3)CC2)OC2(CC2)C1=O] | 12-ethyl-8-(pyridin-3-ylmethyl)-4-oxa-8,12- diazadispiro[2.1.5.3]tridecan-13-one |
| 26 | 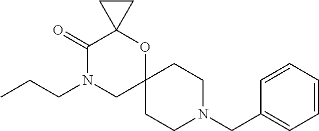 [SMILES: CCCN1CC2(CCN(Cc3ccccc3)CC2)OC2(CC2)C1=O] | 8-benzyl-12-propyl-4-oxa-8,12- diazadispiro[2.1.5.3]tridecan-13-one |
| 27 | 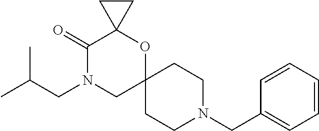 [SMILES: CC(C)CN1CC2(CCN(Cc3ccccc3)CC2)OC2(CC2)C1=O] | 8-benzyl-12-isobutyl-4-oxa-8,12- diazadispiro[2.1.5.3]tridecan-13-one |
| 28 | 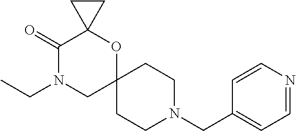 [SMILES: CCN1CC2(CCN(Cc3ccncc3)CC2)OC2(CC2)C1=O] | 12-ethyl-8-(pyridin-4-ylmethyl)-4-oxa-8,12- diazadispiro[2.1.5.3]tridecan-13-one |
| 29 | 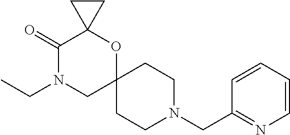 [SMILES: CCN1CC2(CCN(Cc3ccccn3)CC2)OC2(CC2)C1=O] | 12-ethyl-8-(pyridin-2-ylmethyl)-4-oxa-8,12- diazadispiro[2.1.5.3]tridecan-13-one |
| 30 | 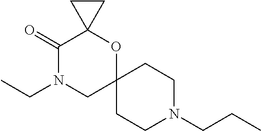 [SMILES: CCCN1CCC2(CC1)CN(CC)C(=O)C1(CC1)O2] | 12-ethyl-8-propyl-4-oxa-8,12- diazadispiro[2.1.5.3]tridecan-13-one |
| 31 | 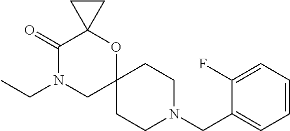 [SMILES: CCN1CC2(CCN(Cc3ccccc3F)CC2)OC2(CC2)C1=O] | 12-ethyl-8-(2-fluorobenzyl)-4-oxa-8,12- diazadispiro[2.1.5.3]tridecan-13-one |
| 32 | 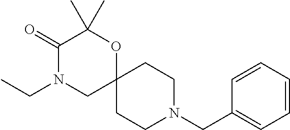 [SMILES: CCN1CC2(CCN(Cc3ccccc3)CC2)OC(C)(C)C1=O] | 9-benzyl-4-ethyl-2,2-dimethyl-1-oxa-4,9- diazaspiro[5.5]undecan-3-one |
| 33 | 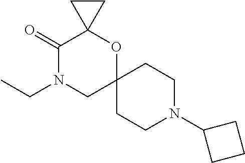 [SMILES: CCN1CC2(CCN(C3CCC3)CC2)OC2(CC2)C1=O] | 8-cyclobutyl-12-ethyl-4-oxa-8,12- diazadispiro[2.1.5.3]tridecan-13-one |
| 34 | 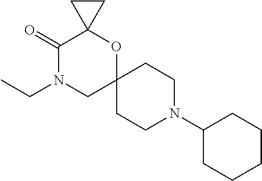 [SMILES: CCN1CC2(CCN(C3CCCCC3)CC2)OC2(CC2)C1=O] | 8-cyclohexyl-12-ethyl-4-oxa-8,12- diazadispiro[2.1.5.3]tridecan-13-one |
| 35 | 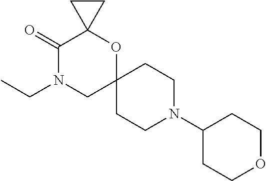 [SMILES: CCN1CC2(CCN(C3CCOCC3)CC2)OC2(CC2)C1=O] | 12-ethyl-8-(tetrahydro-2H-pyran-4-yl)-4- oxa-8,12-diazadispiro[2.1.5.3]tridecan-13- one |
| 36 | 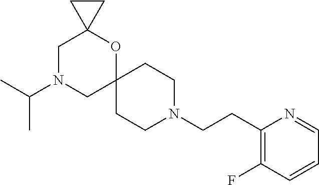 [SMILES: CC(C)N1CC2(CCN(CCc3ncccc3F)CC2)OC2(CC2)C1] | 8-(2-(3-fluoropyridin-2-yl)ethyl)-12- isopropyl-4-oxa-8,12- diazadispiro[2.1.5.3]tridecane |
| 37 | 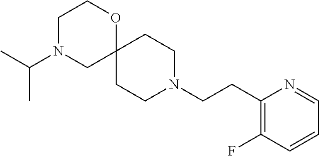 [SMILES: CC(C)N1CCOC2(CCN(CCc3ncccc3F)CC2)C1] | 9-(2-(3-fluoropyridin-2-yl)ethyl)-4-isopropyl- 1-oxa-4,9-diazaspiro[5.5]undecane |
| 38 | 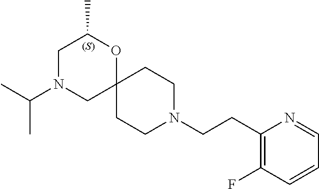 | (S)-9-(2-(3-fluoropyridin-2-yl)ethyl)-4- isopropyl-2-methyl-1-oxa-4,9- diazaspiro[5.5]undecane |
| 39 | 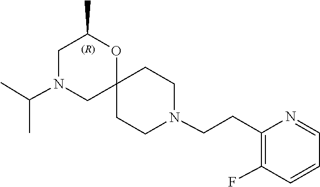 | (R)-9-(2-(3-fluoropyridin-2-yl)ethyl)-4- isopropyl-2-methyl-1-oxa-4,9- diazaspiro[5.5]undecane |
| 40 | 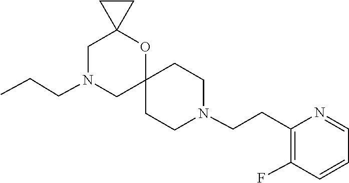 [SMILES: CCCN1CC2(CCN(CCc3ncccc3F)CC2)OC2(CC2)C1] | 8-(2-(3-fluoropyridin-2-yl)ethyl)-12-propyl-4- oxa-8,12-diazadispiro[2.1.5.3]tridecane |
| 41 | 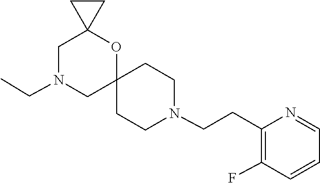 [SMILES: CCN1CC2(CCN(CCc3ncccc3F)CC2)OC2(CC2)C1] | 12-ethyl-8-(2-(3-fluoropyridin-2-yl)ethyl)-4- oxa-8,12-diazadispiro[2.1.5.3]tridecane |
| 42 | 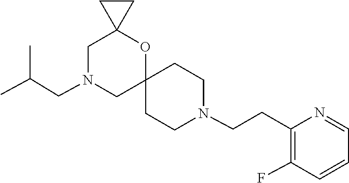 [SMILES: CC(C)CN1CC2(CCN(CCc3ncccc3F)CC2)OC2(CC2)C1] | 8-(2-(3-fluoropyridin-2-yl)ethyl)-12-isobutyl- 4-oxa-8,12-diazadispiro[2.1.5.3]tridecane |
| 43 | 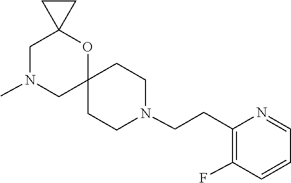 [SMILES: CN1CC2(CCN(CCc3ncccc3F)CC2)OC2(CC2)C1] | 8-(2-(3-fluoropyridin-2-yl)ethyl)-12-methyl- 4-oxa-8,12-diazadispiro[2.1.5.3]tridecane |
| 44 | 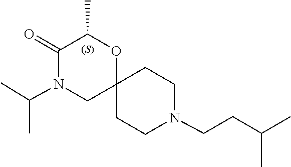 | (S)-9-isopentyl-4-isopropyl-2-methyl-1-oxa- 4,9-diazaspiro[5.5]undecan-3-one |
| 45 | 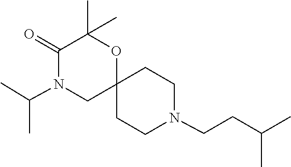 [SMILES: CC(C)CCN1CCC2(CC1)CN(C(C)C)C(=O)C(C)(C)O2] | 9-isopentyl-4-isopropyl-2,2-dimethyl-1-oxa- 4,9-diazaspiro[5.5]undecan-3-one |
| 46 | 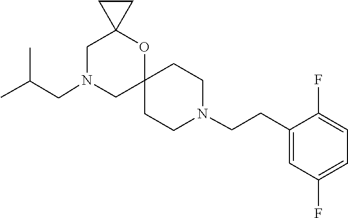 [SMILES: CC(C)CN1CC2(CCN(CCc3cc(F)ccc3F)CC2)OC2(CC2)C1] | 8-(2,5-difluorophenethyl)-12-isobutyl-4- oxa-8,12-diazadispiro[2.1.5.3]tridecane |
| 47 | 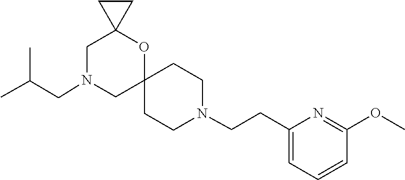 [SMILES: COc1cccc(CCN2CCC3(CC2)CN(CC(C)C)CC2(CC2)O3)n1] | 12-isobutyl-8-(2-(6-methoxypyridin-2- yl)ethyl)-4-oxa-8,12- diazadispiro[2.1.5.3]tridecane |
| 48 | 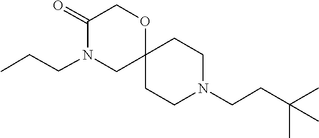 [SMILES: CCCN1CC2(CCN(CCC(C)(C)C)CC2)OCC1=O] | 9-(3,3-dimethylbutyl)-4-propyl-1-oxa-4,9- diazaspiro[5.5]undecan-3-one |
| 49 | 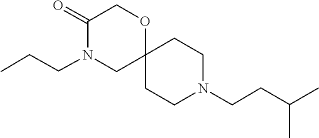 [SMILES: CCCN1CC2(CCN(CCC(C)C)CC2)OCC1=O] | 9-isopentyl-4-propyl-1-oxa-4,9- diazaspiro[5.5]undecan-3-one |
| 50 | 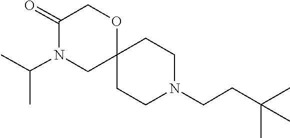 [SMILES: CC(C)N1CC2(CCN(CCC(C)(C)C)CC2)OCC1=O] | 9-(3,3-dimethylbutyl)-4-isopropyl-1-oxa- 4,9-diazaspiro[5.5]undecan-3-one |
| 51 | 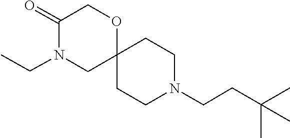 [SMILES: CCN1CC2(CCN(CCC(C)(C)C)CC2)OCC1=O] | 9-(3,3-dimethylbutyl)-4-ethyl-1-oxa-4,9- diazaspiro[5.5]undecan-3-one |
| 52 | 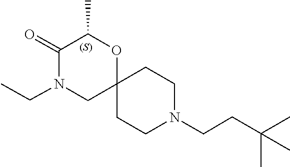 | (S)-9-(3,3-dimethylbutyl)-4-ethyl-2-methyl- 1-oxa-4,9-diazaspiro[5.5]undecan-3-one |
| 53 | 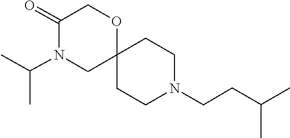 [SMILES: CC(C)CCN1CCC2(CC1)CN(C(C)C)C(=O)CO2] | 9-isopentyl-4-isopropyl-1-oxa-4,9- diazaspiro[5.5]undecan-3-one |
| 54 | 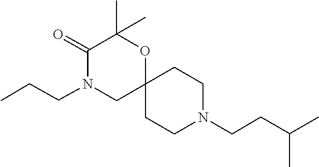 [SMILES: CCCN1CC2(CCN(CCC(C)C)CC2)OC(C)(C)C1=O] | 9-isopentyl-2,2-dimethyl-4-propyl-1-oxa- 4,9-diazaspiro[5.5]undecan-3-one |
| 55 | 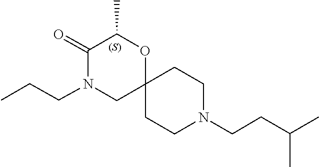 | (S)-9-isopentyl-2-methyl-4-propyl-1-oxa- 4,9-diazaspiro[5.5]undecan-3-one |
| 56 | 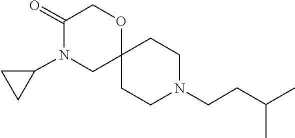 [SMILES: CC(C)CCN1CCC2(CC1)CN(C1CC1)C(=O)CO2] | 4-cyclopropyl-9-isopentyl-1-oxa-4,9- diazaspiro[5.5]undecan-3-one |
| 57 | 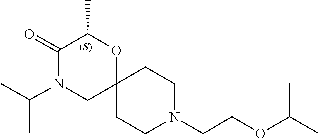 | (S)-9-(2-isopropoxyethyl)-4-isopropyl-2- methyl-1-oxa-4,9-diazaspiro[5.5]undecan- 3-one |
| 58 | 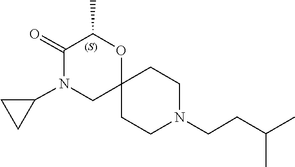 | (S)-4-cyclopropyl-9-isopentyl-2-methyl-1- oxa-4,9-diazaspiro[5.5]undecan-3-one |
| 59 | 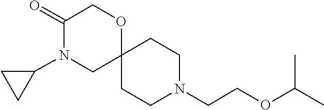 [SMILES: CC(C)OCCN1CCC2(CC1)CN(C1CC1)C(=O)CO2] | 4-cyclopropyl-9-(2-isopropoxyethyl)-1-oxa- 4,9-diazaspiro[5.5]undecan-3-one |
| 60 | 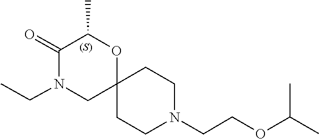 | (S)-4-ethyl-9-(2-isopropoxyethyl)-2-methyl- 1-oxa-4,9-diazaspiro[5.5]undecan-3-one |
| 61 | 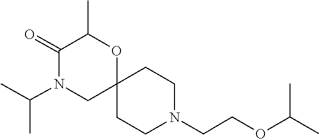 [SMILES: CC(C)OCCN1CCC2(CC1)CN(C(C)C)C(=O)C(C)O2] | 9-(2-isopropoxyethyl)-4-isopropyl-2-methyl- 1-oxa-4,9-diazaspiro[5.5]undecan-3-one |
| 62 | 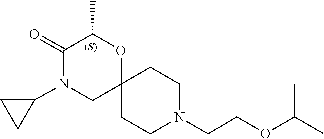 | (S)-4-cyclopropyl-9-(2-isopropoxyethyl)-2- methyl-1-oxa-4,9-diazaspiro[5.5]undecan- 3-one |
| 63 | 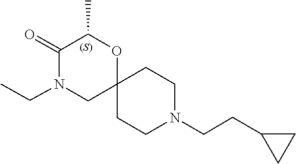 | (S)-9-(2-cyclopropylethyl)-4-ethyl-2-methyl- 1-oxa-4,9-diazaspiro[5.5]undecan-3-one |
| 64 | 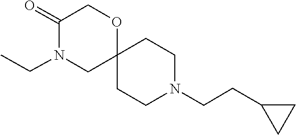 [SMILES: CCN1CC2(CCN(CCC3CC3)CC2)OCC1=O] | 9-(2-cyclopropylethyl)-4-ethyl-1-oxa-4,9- diazaspiro[5.5]undecan-3-one |
| 65 | 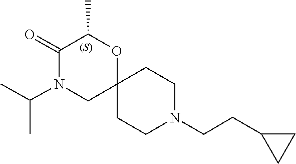 | (S)-9-(2-cyclopropylethyl)-4-isopropyl-2- methyl-1-oxa-4,9-diazaspiro[5.5]undecan- 3-one |
| 66 | 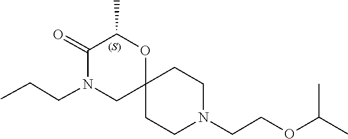 | (S)-9-(2-isopropoxyethyl)-2-methyl-4- propyl-1-oxa-4,9-diazaspiro[5.5]undecan- 3-one |
| 67 | 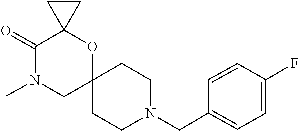 [SMILES: CN1CC2(CCN(Cc3ccc(F)cc3)CC2)OC2(CC2)C1=O] | 8-(4-fluorobenzyl)-12-methyl-4-oxa-8,12- diazadispiro[2.1.5.3]tridecan-13-one |
| 68 | 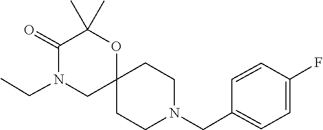 [SMILES: CCN1CC2(CCN(Cc3ccc(F)cc3)CC2)OC(C)(C)C1=O] | 4-ethyl-9-(4-fluorobenzyl)-2,2-dimethyl-1- oxa-4,9-diazaspiro[5.5]undecan-3-one |
| 69 | 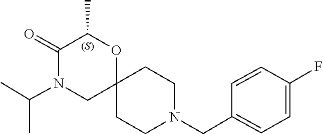 | (S)-9-(4-fluorobenzyl)-4-isopropyl-2- methyl-1-oxa-4,9-diazaspiro[5.5]undecan- 3-one |
| 70 | 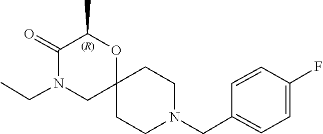 | (R)-4-ethyl-9-(4-fluorobenzyl)-2-methyl-1- oxa-4,9-diazaspiro[5.5]undecan-3-one |
| 71 | 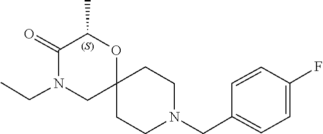 | (S)-4-ethyl-9-(4-fluorobenzyl)-2-methyl-1- oxa-4,9-diazaspiro[5.5]undecan-3-one |
| 72 | 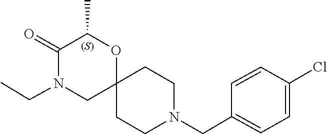 | (S)-9-(4-chlorobenzyl)-4-ethyl-2-methyl-1- oxa-4,9-diazaspiro[5.5]undecan-3-one |
| 73 | 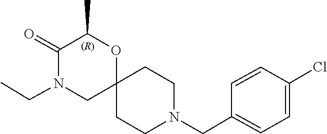 | (R)-9-(4-chlorobenzyl)-4-ethyl-2-methyl-1- oxa-4,9-diazaspiro[5.5]undecan-3-one |
| 74 | 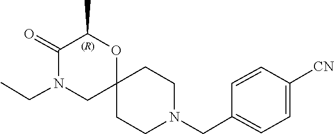 | (R)-4-((4-ethyl-2-methyl-3-oxo-1-oxa-4,9- diazaspiro[5.5]undecan-9- yl)methyl)benzonitrile |
| 75 | 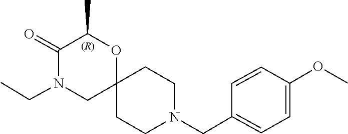 | (R)-4-ethyl-9-(4-methoxybenzyl)-2-methyl- 1-oxa-4,9-diazaspiro[5.5]undecan-3-one |
| 76 | 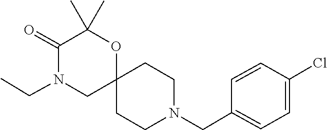 [SMILES: CCN1CC2(CCN(Cc3ccc(Cl)cc3)CC2)OC(C)(C)C1=O] | 9-(4-chlorobenzyl)-4-ethyl-2,2-dimethyl-1- oxa-4,9-diazaspiro[5.5]undecan-3-one |
| 77 | 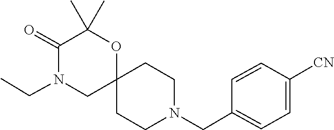 [SMILES: CCN1CC2(CCN(Cc3ccc(C#N)cc3)CC2)OC(C)(C)C1=O] | 4-((4-ethyl-2,2-dimethyl-3-oxo-1-oxa-4,9- diazaspiro[5.5]undecan-9- yl)methyl)benzonitrile |
| 78 | 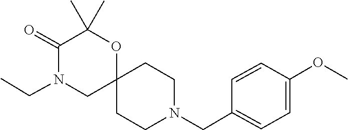 [SMILES: CCN1CC2(CCN(Cc3ccc(OC)cc3)CC2)OC(C)(C)C1=O] | 4-ethyl-9-(4-methoxybenzyl)-2,2-dimethyl- 1-oxa-4,9-diazaspiro[5.5]undecan-3-one |
| 79 | 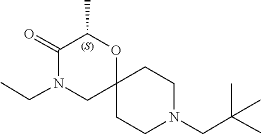 | (S)-4-ethyl-2-methyl-9-neopentyl-1-oxa- 4,9-diazaspiro[5.5]undecan-3-one |
| 80 | 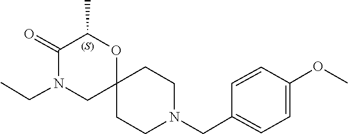 | (S)-4-ethyl-9-(4-methoxybenzyl)-2-methyl- 1-oxa-4,9-diazaspiro[5.5]undecan-3-one |
| 81 | 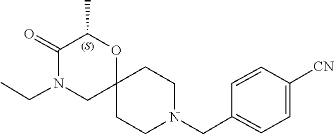 | (S)-4-((4-ethyl-2-methyl-3-oxo-1-oxa-4,9- diazaspiro[5.5]undecan-9- yl)methyl)benzonitrile |
| 82 | 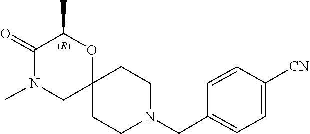 | (R)-4-((2,4-dimethyl-3-oxo-1-oxa-4,9- diazaspiro[5.5]undecan-9- yl)methyl)benzonitrile |
| 83 | 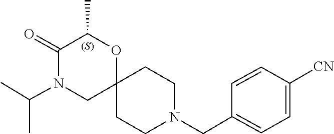 | (S)-4-((4-isopropyl-2-methyl-3-oxo-1-oxa- 4,9-diazaspiro[5.5]undecan-9- yl)methyl)benzonitrile |
| 84 | 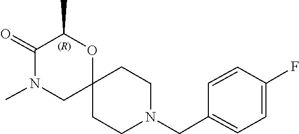 | (R)-9-(4-fluorobenzyl)-2,4-dimethyl-1-oxa- 4,9-diazaspiro[5.5]undecan-3-one |
| 85 | 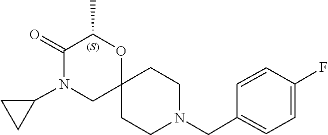 | (S)-4-cyclopropyl-9-(4-fluorobenzyl)-2- methyl-1-oxa-4,9-diazaspiro[5.5]undecan- 3-one |
| 86 | 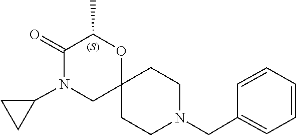 | (S)-9-benzyl-4-cyclopropyl-2-methyl-1-oxa- 4,9-diazaspiro[5.5]undecan-3-one |
| 87 | 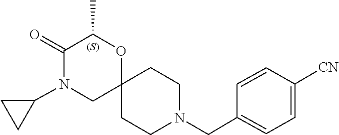 | (S)-4-((4-cyclopropyl-2-methyl-3-oxo-1- oxa-4,9-diazaspiro[5.5]undecan-9- yl)methyl)benzonitrile |
| 88 | 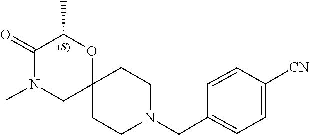 | (S)-4-((2,4-dimethyl-3-oxo-1-oxa-4,9- diazaspiro[5.5]undecan-9- yl)methyl)benzonitrile |
| 89 | 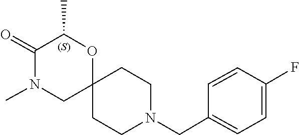 | (S)-9-(4-fluorobenzyl)-2,4-dimethyl-1-oxa- 4,9-diazaspiro[5.5]undecan-3-one |
| 90 | 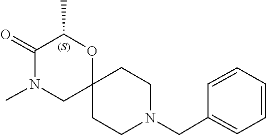 | (S)-9-benzyl-2,4-dimethyl-1-oxa-4,9- diazaspiro[5.5]undecan-3-one |
| 91 | 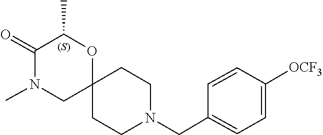 | (S)-2,4-dimethyl-9-(4- (trifluoromethoxy)benzyl)-1-oxa-4,9- diazaspiro[5.5]undecan-3-one |
| 92 | 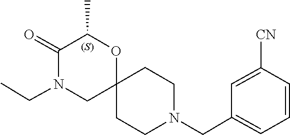 | (S)-3-((4-ethyl-2-methyl-3-oxo-1-oxa-4,9- diazaspiro[5.5]undecan-9- yl)methyl)benzonitrile |
| 93 | 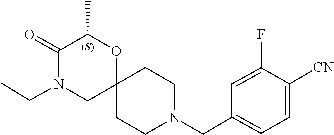 | (S)-4-((4-ethyl-2-methyl-3-oxo-1-oxa-4,9- diazaspiro[5.5]undecan-9-yl)methyl)-2- fluorobenzonitrile |
| 94 | 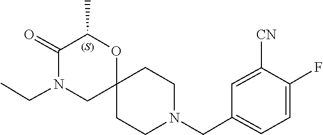 | (S)-5-((4-ethyl-2-methyl-3-oxo-1-oxa-4,9- diazaspiro[5.5]undecan-9-yl)methyl)-2- fluorobenzonitrile |
| 95 | 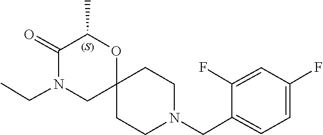 | (S)-9-(2,4-difluorobenzyl)-4-ethyl-2-methyl- 1-oxa-4,9-diazaspiro[5.5]undecan-3-one |
| 96 | 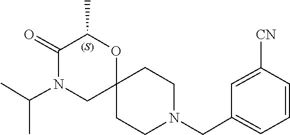 | (S)-3-((4-isopropyl-2-methyl-3-oxo-1-oxa- 4,9-diazaspiro[5.5]undecan-9- yl)methyl)benzonitrile |
| 97 | 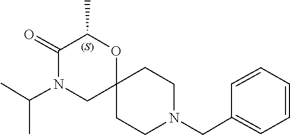 | (S)-9-benzyl-4-isopropyl-2-methyl-1-oxa- 4,9-diazaspiro[5.5]undecan-3-one |
| 98 | 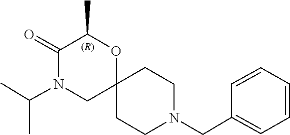 | (R)-9-benzyl-4-isopropyl-2-methyl-1-oxa- 4,9-diazaspiro[5.5]undecan-3-one |
| 99 | 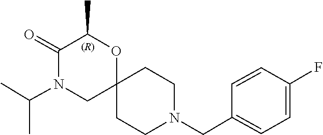 | (R)-9-(4-fluorobenzyl)-4-isopropyl-2- methyl-1-oxa-4,9-diazaspiro[5.5]undecan- 3-one |
| 100 | 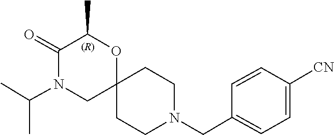 | (R)-4-((4-isopropyl-2-methyl-3-oxo-1-oxa- 4,9-diazaspiro[5.5]undecan-9- yl)methyl)benzonitrile |
| 101 | 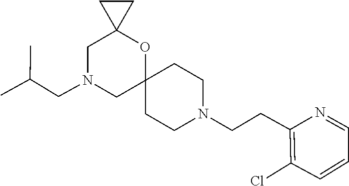 [SMILES: CC(C)CN1CC2(CCN(CCc3ncccc3Cl)CC2)OC2(CC2)C1] | 8-(2-(3-chloropyridin-2-yl)ethyl)-12- isobutyl-4-oxa-8,12- diazadispiro[2.1.5.3]tridecane |
| 102 | 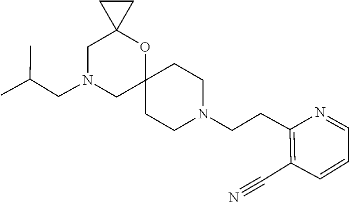 [SMILES: CC(C)CN1CC2(CCN(CCc3ncccc3C#N)CC2)OC2(CC2)C1] | 2-(2-(12-isobutyl-4-oxa-8,12- diazadispiro[2.1.5.3]tridecan-8- yl)ethyl)nicotinonitrile |
| 103 | 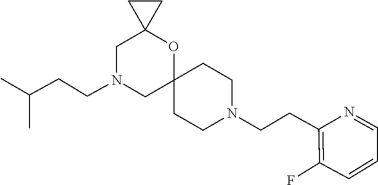 [SMILES: CC(C)CCN1CC2(CCN(CCc3ncccc3F)CC2)OC2(CC2)C1] | 8-(2-(3-fluoropyridin-2-yl)ethyl)-12- isopentyl-4-oxa-8,12- diazadispiro[2.1.5.3]tridecane |
| 104 | 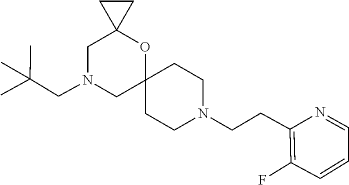 [SMILES: CC(C)(C)CN1CC2(CCN(CCc3ncccc3F)CC2)OC2(CC2)C1] | 8-(2-(3-fluoropyridin-2-yl)ethyl)-12- neopentyl-4-oxa-8,12- diazadispiro[2.1.5.3]tridecane |
| 105 | 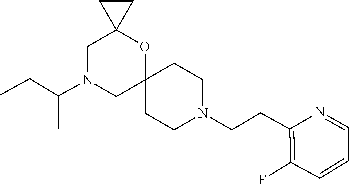 [SMILES: CCC(C)N1CC2(CCN(CCc3ncccc3F)CC2)OC2(CC2)C1] | 12-(sec-butyl)-8-(2-(3-fluoropyridin-2- yl)ethyl)-4-oxa-8,12- diazadispiro[2.1.5.3]tridecane |
| 106 | 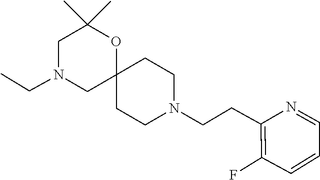 [SMILES: CCN1CC(C)(C)OC2(CCN(CCc3ncccc3F)CC2)C1] | 4-ethyl-9-(2-(3-fluoropyridin-2-yl)ethyl)-2,2- dimethyl-1-oxa-4,9- diazaspiro[5.5]undecane |
| 107 | 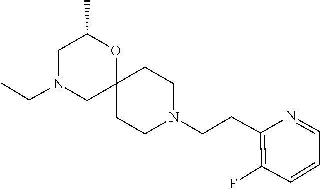 [SMILES: CCN1C[C@H](C)OC2(CCN(CCc3ncccc3F)CC2)C1] | (S)-4-ethyl-9-(2-(3-fluoropyridin-2-yl)ethyl)- 2-methyl-1-oxa-4,9- diazaspiro[5.5]undecane |
| 108 | 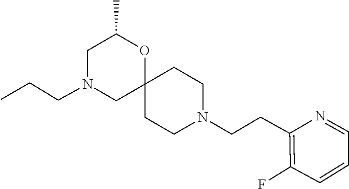 [SMILES: CCCN1C[C@H](C)OC2(CCN(CCc3ncccc3F)CC2)C1] | (S)-9-(2-(3-fluoropyridin-2-yl)ethyl)-2- methyl-4-propyl-1-oxa-4,9- diazaspiro[5.5]undecane |
| 109 | 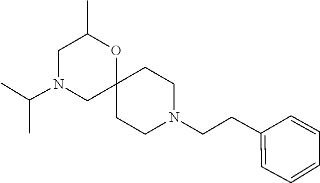 [SMILES: CC1CN(C(C)C)CC2(CCN(CCc3ccccc3)CC2)O1] | 4-Isopropyl-2-methyl-9-phenethyl-1-oxa- 4,9-diazaspiro[5.5]undecane |
| 110 | 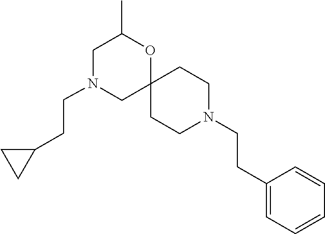 [SMILES: CC1CN(CCC2CC2)CC2(CCN(CCc3ccccc3)CC2)O1] | 4-(2-cyclopropylethyl)-2-methyl-9- phenethyl-1-oxa-4,9- diazaspiro[5.5]undecane |
| 111 | 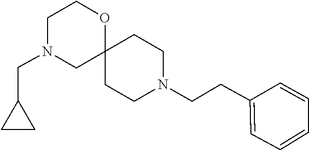 [SMILES: c1ccc(CCN2CCC3(CC2)CN(CC2CC2)CCO3)cc1] | 4-(cyclopropylmethyl)-9-phenethyl-1-oxa- 4,9-diazaspiro[5.5]undecane |
| 112 | 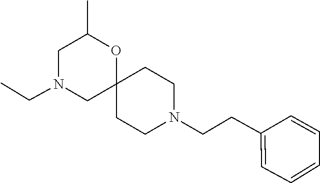 [SMILES: CCN1CC(C)OC2(CCN(CCc3ccccc3)CC2)C1] | 4-Ethyl-2-methyl-9-phenethyl-1-oxa-4,9- diazaspiro[5.5]undecane |
| 113 | 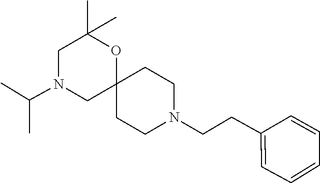 [SMILES: CC(C)N1CC(C)(C)OC2(CCN(CCc3ccccc3)CC2)C1] | 4-isopropyl-2,2-dimethyl-9-phenethyl-1- oxa-4,9-diazaspiro[5.5]undecane |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) for use according to the invention are selected from

| NUMBER | STRUCTURE | CHEMICAL NAME |
|---|---|---|
| 16 | 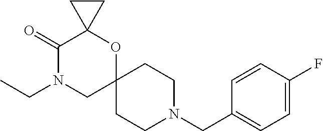 [SMILES: CCN1CC2(CCN(Cc3ccc(F)cc3)CC2)OC2(CC2)C1=O] | 12-ethyl-8-(4-fluorobenzyl)-4-oxa-8,12- diazadispiro[2.1.5.3]tridecan-13-one |
| 17 | 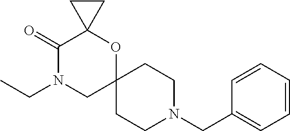 [SMILES: CCN1CC2(CCN(Cc3ccccc3)CC2)OC2(CC2)C1=O] | 8-benzyl-12-ethyl-4-oxa-8,12- diazadispiro[2.1.5.3]tridecan-13-one |
| 18 | 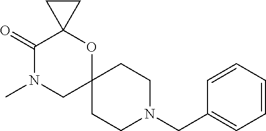 [SMILES: CN1CC2(CCN(Cc3ccccc3)CC2)OC2(CC2)C1=O] | 8-benzyl-12-methyl-4-oxa-8,12- diazadispiro[2.1.5.3]tridecan-13-one |
| 19 | 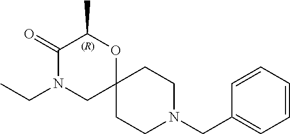 | (R)-9-benzyl-4-ethyl-2-methyl-1-oxa-4,9- diazaspiro[5.5]undecan-3-one |
| 20 | 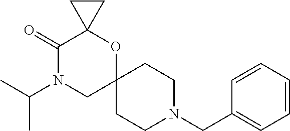 [SMILES: CC(C)N1CC2(CCN(Cc3ccccc3)CC2)OC2(CC2)C1=O] | 8-benzyl-12-isopropyl-4-oxa-8,12- diazadispiro[2.1.5.3]tridecan-13-one |
| 22 | 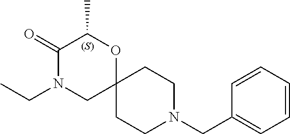 | (S)-9-benzyl-4-ethyl-2-methyl-1-oxa-4,9- diazaspiro[5.5]undecan-3-one |
| 23 | 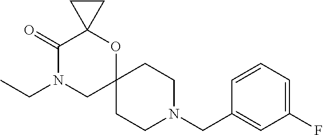 [SMILES: CCN1CC2(CCN(Cc3cccc(F)c3)CC2)OC2(CC2)C1=O] | 12-ethyl-8-(3-fluorobenzyl)-4-oxa-8,12- diazadispiro[2.1.5.3]tridecan-13-one |
| 24 | 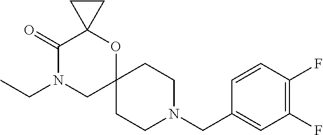 [SMILES: CCN1CC2(CCN(Cc3ccc(F)c(F)c3)CC2)OC2(CC2)C1=O] | 8-(3,4-difluorobenzyl)-12-ethyl-4-oxa-8,12- diazadispiro[2.1.5.3]tridecan-13-one |
| 25 | 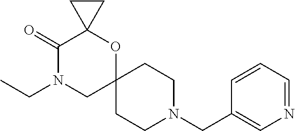 [SMILES: CCN1CC2(CCN(Cc3cccnc3)CC2)OC2(CC2)C1=O] | 12-ethyl-8-(pyridin-3-ylmethyl)-4-oxa-8,12- diazadispiro[2.1.5.3]tridecan-13-one |
| 26 | 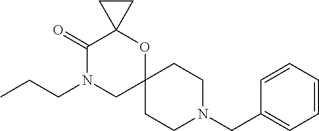 [SMILES: CCCN1CC2(CCN(Cc3ccccc3)CC2)OC2(CC2)C1=O] | 8-benzyl-12-propyl-4-oxa-8,12- diazadispiro[2.1.5.3]tridecan-13-one |
| 27 | 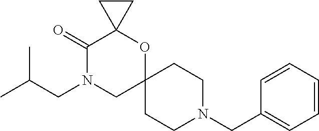 [SMILES: CC(C)CN1CC2(CCN(Cc3ccccc3)CC2)OC2(CC2)C1=O] | 8-benzyl-12-isobutyl-4-oxa-8,12- diazadispiro[2.1.5.3]tridecan-13-one |
| 28 | 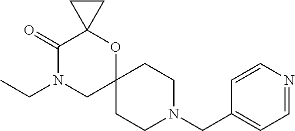 [SMILES: CCN1CC2(CCN(Cc3ccncc3)CC2)OC2(CC2)C1=O] | 12-ethyl-8-(pyridin-4-ylmethyl)-4-oxa-8,12- diazadispiro[2.1.5.3]tridecan-13-one |
| 29 | 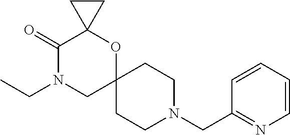 [SMILES: CCN1CC2(CCN(Cc3ccccn3)CC2)OC2(CC2)C1=O] | 12-ethyl-8-(pyridin-2-ylmethyl)-4-oxa-8,12- diazadispiro[2.1.5.3]tridecan-13-one |
| 31 | 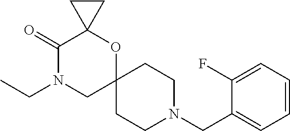 [SMILES: CCN1CC2(CCN(Cc3ccccc3F)CC2)OC2(CC2)C1=O] | 12-ethyl-8-(2-fluorobenzyl)-4-oxa-8,12- diazadispiro[2.1.5.3]tridecan-13-one |
| 32 | 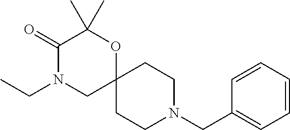 [SMILES: CCN1CC2(CCN(Cc3ccccc3)CC2)OC(C)(C)C1=O] | 9-benzyl-4-ethyl-2,2-dimethyl-1-oxa-4,9- diazaspiro[5.5]undecan-3-one |
| 67 | 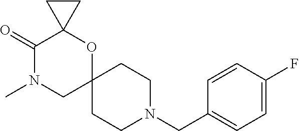 [SMILES: CN1CC2(CCN(Cc3ccc(F)cc3)CC2)OC2(CC2)C1=O] | 8-(4-fluorobenzyl)-12-methyl-4-oxa-8,12- diazadispiro[2.1.5.3]tridecan-13-one |
| 68 | 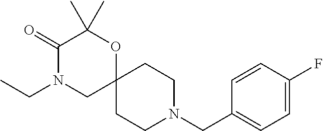 [SMILES: CCN1CC2(CCN(Cc3ccc(F)cc3)CC2)OC(C)(C)C1=O] | 4-ethyl-9-(4-fluorobenzyl)-2,2-dimethyl-1- oxa-4,9-diazaspiro[5.5]undecan-3-one |
| 69 | 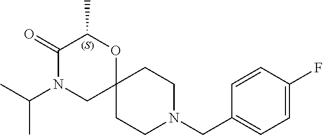 | (S)-9-(4-fluorobenzyl)-4-isopropyl-2-methyl- 1-oxa-4,9-diazaspiro[5.5]undecan-3-one |
| 70 | 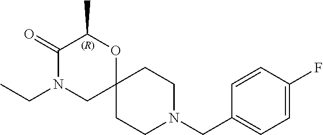 | (R)-4-ethyl-9-(4-fluorobnezyl)-2-methyl-1- oxa-4,9-diazaspiro[5.5]undecan-3-one |
| 71 | 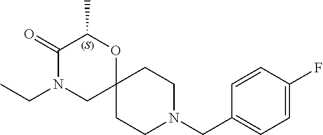 | (S)-4-ethyl-9-(4-fluorobenzyl)-2-methyl-1- oxa-4,9-diazaspiro[5.5]undecan-3-one |
| 72 | 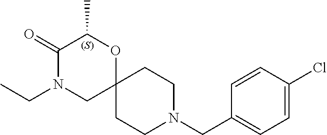 | (S)-9-(4-chlorobenzyl)-4-ethyl-2-methyl-1- oxa-4,9-diazaspiro[5.5]undecan-3-one |
| 73 | 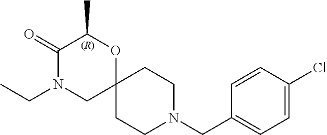 | (R)-9-(4-chlorobenzyl)-4-ethyl-2-methyl-1- oxa-4,9-diazaspiro[5.5]undecan-3-one |
| 74 | 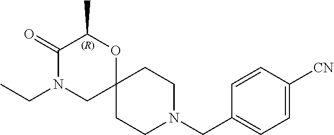 | (R)-4-((4-ethyl-2-methyl-3-oxo-1-oxa-4,9- diazaspiro[5.5]undecan-9- yl)methyl)benzonitrile |
| 75 | 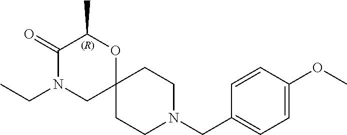 | (R)-4-ethyl-9-(4-methoxybenzyl)-2-methyl- 1-oxa-4,9-diazaspiro[5.5]undecan-3-one |
| 76 | 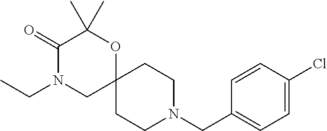 [SMILES: CCN1CC2(CCN(Cc3ccc(Cl)cc3)CC2)OC(C)(C)C1=O] | 9-(4-chlorobenzyl)-4-ethyl-2,2-dimethyl-1- oxa-4,9-diazaspiro[5.5]undecan-3-one |
| 77 | 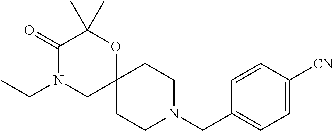 [SMILES: CCN1CC2(CCN(Cc3ccc(C#N)cc3)CC2)OC(C)(C)C1=O] | 4-((4-ethyl-2,2-dimethyl-3-oxo-1-oxa-4,9- diazaspiro[5.5]undecan-9- yl)methyl)benzonitrile |
| 78 | 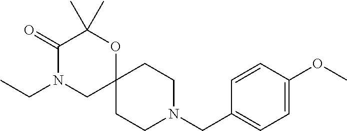 [SMILES: CCN1CC2(CCN(Cc3ccc(OC)cc3)CC2)OC(C)(C)C1=O] | 4-ethyl-9-(4-methoxybenzyl)-2,2-dimethyl- 1-oxa-4,9-diazaspiro[5.5]undecan-3-one |
| 80 | 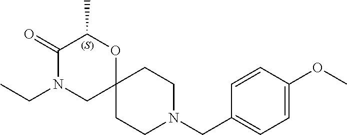 | (S)-4-ethyl-9-(4-methoxybenzyl)-2-methyl- 1-oxa-4,9-diazaspiro[5.5]undecan-3-one |
| 81 | 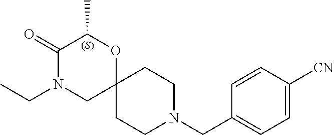 | (S)-4-((4-ethyl-2-methyl-3-oxo-1-oxa-4,9- diazaspiro[5.5]undecan-9- yl)methyl)benzonitrile |
| 82 | 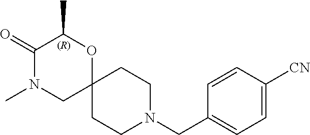 | (R)-4-((2,4-dimethyl-3-oxo-1-oxa-4,9- diazaspiro[5.5]undecan-9- yl)methyl)benzonitrile |
| 83 | 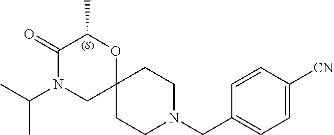 | (S)-4-((4-isopropyl-2-methyl-3-oxo-1-oxa- 4,9-diazaspiro[5.5]undecan-9- yl)methyl)benzonitrile |
| 84 | 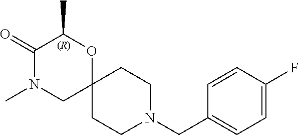 | (R)-9-(4-fluorobenzyl)-2,4-dimethyl-1-oxa- 4,9-diazaspiro[5.5]undecan-3-one |
| 85 | 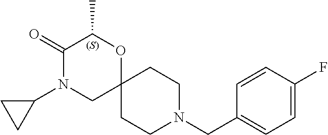 | (S)-4-cyclopropyl-9-(4-fluorobenzyl)-2- methyl-1-oxa-4,9-diazaspiro[5.5]undecan- 3-one |
| 86 | 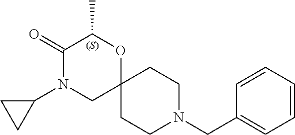 | (S)-9-benzyl-4-cyclopropyl-2-methyl-1-oxa- 4,9-diazaspiro[5.5]undecan-3-one |
| 87 | 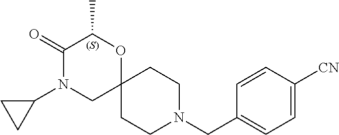 | (S)-4-((4-cyclopropyl-2-methyl-3-oxo-1- oxa-4,9-diazaspiro[5.5]undecan-9- yl)methyl)benzonitrile |
| 88 | 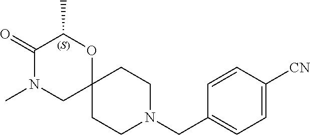 | (S)-4-((2,4-dimethyl-3-oxo-1-oxa-4,9- diazaspiro[5.5]undecan-9- yl)methyl)benzonitrile |
| 89 | 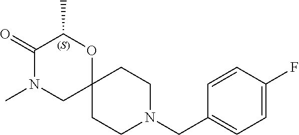 | (S)-9-(4-fluorobenzyl)-2,4-dimethyl-1-oxa- 4,9-diazaspiro[5.5]undecan-3-one |
| 90 | 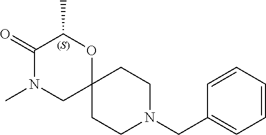 | (S)-9-benzyl-2,4-dimethyl-1-oxa-4,9- diazaspiro[5.5]undecan-3-one |
| 91 | 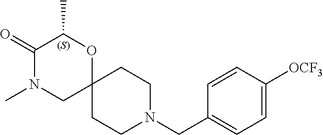 | (S)-2,4-dimethyl-9-(4- (trifluoromethoxy)benzyl)-1-oxa-4,9- diazaspiro[5.5]undecan-3-one |
| 92 | 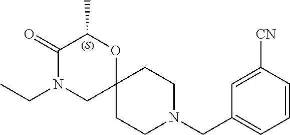 | (S)-3-((4-ethyl-2-methyl-3-oxo-1-oxa-4,9- diazaspiro[5.5]undecan-9- yl)methyl)benzonitrile |
| 93 | 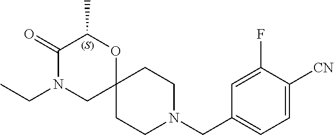 | (S)-4-((4-ethyl-2-methyl-3-oxo-1-oxa-4,9- diazaspiro[5.5]undecan-9-yl)methyl)-2- fluorobenzonitrile |
| 94 | 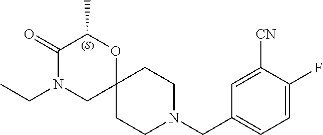 | (S)-5-((4-ethyl-2-methyl-3-oxo-1-oxa-4,9- diazaspiro[5.5]undecan-9-yl)methyl)-2- fluorobenzonitrile |
| 95 | 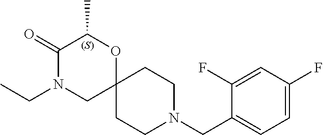 | (S)-9-(2,4-difluorobenzyl)-4-ethyl-2-methyl- 1-oxa-4,9-diazaspiro[5.5]undecan-3-one |
| 96 | 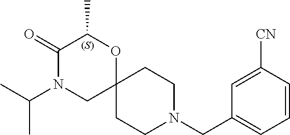 | (S)-3-((4-isopropyl-2-methyl-3-oxo-1-oxa- 4,9-diazaspiro[5.5]undecan-9- yl)methyl)benzonitriile |
| 97 | 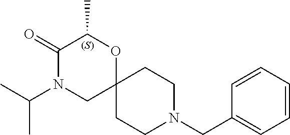 | (S)-9-benzyl-4-isopropyl-2-methyl-1-oxa- 4,9-diazaspiro[5.5]undecan-3-one |
| 98 | 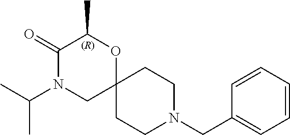 | (R)-9-benzyl-4-isopropyl-2-methyl-1-oxa- 4,9-diazaspiro[5.5]undecan-3-one |
| 99 | 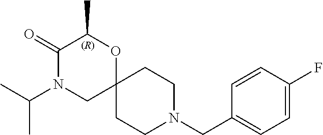 | (R)-9-(4-fluorobenzyl)-4-isopropyl-2- methyl-1-oxa-4,9-diazaspiro[5.5]undecan- 3-one |
| 100 | 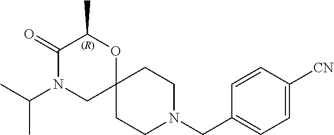 | (R)-4-((4-isopropyl-2-methyl-3-oxo-1-oxa- 4,9-diazaspiro[5.5]undecan-9- yl)methyl)benzonitrile |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred further embodiment, the compounds of the general Formula (I) for use according to the invention are selected from

| NUMBER | STRUCTURE | CHEMICAL NAME |
|---|---|---|
| 89 | 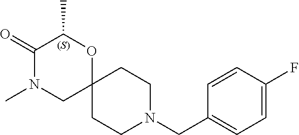 | (S)-9-(4-fluorobenzyl)-2,4-dimethyl-1-oxa- 4,9-diazaspiro[5.5]undecan-3-one |
| 93 | 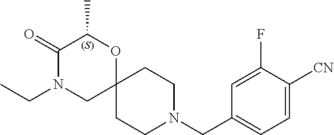 | (S)-4-((4-ethyl-2-methyl-3-oxo-1-oxa-4,9- diazaspiro[5.5]undecan-9-yl)methyl)-2- fluorobenzonitrile |

optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound of general Formula (I) for use according to the invention, and in relation to R₁ of any of the embodiments of the description,
the cycloalkyl in R₁ if substituted, is substituted with one or more substituent/s selected from halogen, —R₁₁, —OR₁₁, —NO₂, —NR₁₁R_{11′″}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OH, —NR₁₁S(O)₂NR_{11′}R_{11″} and C(CH₃)₂OR₁₁.

In a preferred embodiment of the compound of general Formula (I) for use according to the invention, and in relation to R₁ of any of the embodiments of the description,
the cycloalkyl in R₁, if substituted, may also be substituted with

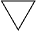

or ═O.

In a preferred embodiment of the compound of general Formula (I) for use according to the invention, and in relation to R₁ of any of the embodiments of the description, the alkyl, alkenyl or alkynyl in R₁, if substituted, is substituted with one or more substituent/s selected from —OR₁₁, halogen, —CN, haloalkyl, haloalkoxy and —NR₁₁R_{11″″}.

In a preferred embodiment of the compound of general Formula (I) for use according to the invention, and in relation to R₂ of any of the embodiments of the description, the cycloalkyl, aryl or heterocyclyl in R₂, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OH, —NR₁₂S(O)₂NR_{12′}R_{12″} and C(CH₃)₂OR₁₂.

In a preferred embodiment of the compound of general Formula (I) for use according to the invention, and in relation to R₂ of any of the embodiments of the description, the cycloalkyl, aryl or heterocyclyl in R₂, if substituted, is substituted with one or more substituent/s selected from halogen, —OR₁₂, —CN and haloalkoxy.

In a preferred embodiment of the compound of general Formula (I) for use according to the invention, and in relation to R₂ of any of the embodiments of the description, the cycloalkyl, aryl or heterocyclyl in R₂, if substituted, is substituted with one or more substituent/s selected from halogen and CN.

In a preferred embodiment of the compound of general Formula (I) for use according to the invention, and in relation to R₂ of any of the embodiments of the description, the cycloalkyl or non-aromatic heterocyclyl in R₂, if substituted, may also be substituted with

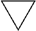

### or ═O.

In a preferred embodiment of the compound of general Formula (I) for use according to the invention, and in relation to R₂ of any of the embodiments of the description, the alkyl, alkenyl or alkynyl in R₂, if substituted, is substituted with one or more substituent/s selected from —OR₁₂, halogen, —CN, haloalkyl, haloalkoxy and —NR₁₂R_{12′″}.

In a preferred embodiment of the compound of general Formula (I) for use according to the invention, of any of the embodiments of the description, the alkyl, alkenyl or alkynyl, other than those defined in R₁ or R₂, if substituted, is substituted with one or more substituent/s selected from —OR₁₃, halogen, —CN, haloalkyl, haloalkoxy and —NR₁₃R_{13′″}.

In a preferred embodiment of the compound of general Formula (I) for use according to the invention of any of the embodiments of the description, the aryl, heterocyclyl or cycloalkyl other than those defined in R₁ or R₂, if substituted, is substituted with one or more substituent/s selected from halogen, —R₁₄, —OR₁₄, —NO₂, —NR₁₄R_{14′″}, NR₁₄C(O)R_{14′}, —NR₁₄S(O)₂R_{14′}, —S(O)₂NR₁₄R_{14′}, —NR₁₄C(O)NR_{14′}R_{14″}, —SR₁₄, —S(O)R₁₄, S(O)₂R₁₄, —CN, haloalkyl, haloalkoxy, —C(O)OR₁₄, —C(O)NR₁₄R_{14′}, —OCH₂CH₂OH, —NR₁₄S(O)₂NR_{14′}R_{14″} and C(CH₃)₂OR₁₄.

In a preferred embodiment of the compound of general Formula (I) for use according to the invention, and in relation to the cycloalkyl, aryl or heterocyclyl other than those defined in R₁ or R₂ of any of the embodiments of the description, the cycloalkyl or non-aromatic heterocyclyl, other than those defined in R₁ or R₂, if substituted, may also be substituted with

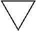

### or ═O.

In a preferred embodiment of the compound of general Formula (I) for use according to the invention,
the halogen is fluorine, chlorine, iodine or bromine, preferably fluorine or chlorine; more preferably fluorine;
optionally in form of one of the stereoisomers, preferably enantiomers or diastereomers, a racemate or in form of a mixture of at least two of the stereoisomers, preferably enantiomers and/or diastereomers, in any mixing ratio, or a corresponding salt thereof, or a corresponding solvate thereof.

In a preferred embodiment of the compound of general Formula (I) for use according to the invention,
the haloalkyl is —CF₃.

In a preferred embodiment of the compound of general Formula (I) for use according to the invention,
the haloalkoxy is —OCF₃.

In an aspect, the present invention relates to neuroprotection or to a method of treatment of motoneuron degeneration comprising administering to a subject in need thereof a therapeutically effective amount of a compound of Formula (I).

In a particular embodiment the present invention relates to neuroprotection comprising administering to a subject in need thereof a therapeutically effective amount of a compound of Formula (I).

In a particular embodiment the present invention relates to a method of treatment of motoneuron degeneration comprising administering to a subject in need thereof a therapeutically effective amount of a compound of Formula (I).

In a particular embodiment the present invention relates to a method of treatment of ALS comprising administering to a subject in need thereof a therapeutically effective amount of a compound of Formula (I).

In a particular embodiment the present invention relates to a method of preventing motoneuron death comprising administering to a subject in need thereof a therapeutically effective amount of a compound of Formula (I).

In a particular embodiment the present invention relates to a method of preventing motoneuron death under chronic excitotoxic stress comprising administering to a subject in need thereof a therapeutically effective amount of a compound of Formula (I).

In a particular embodiment the present invention relates to a method of enhancing motoneuron survival comprising administering to a subject in need thereof a therapeutically effective amount of a compound of Formula (I).

In a particular embodiment the present invention relates to a method of enhancing motoneuron survival after rhizotomy comprising administering to a subject in need thereof a therapeutically effective amount of a compound of Formula (I).

In a particular embodiment the present invention relates to a method of slowing motoneuron degeneration comprising administering to a subject in need thereof a therapeutically effective amount of a compound of Formula (I).

In a particular embodiment the present invention relates to a method of preserving neuromuscular junctions innervation comprising administering to a subject in need thereof a therapeutically effective amount of a compound of Formula (I).

In a particular embodiment the present invention relates to a method of protecting spinal motoneurons comprising administering to a subject in need thereof a therapeutically effective amount of a compound of Formula (I).

In a particular embodiment the present invention relates to a method of reducing astroglial activation comprising administering to a subject in need thereof a therapeutically effective amount of a compound of Formula (I).

In an aspect, the present invention relates to a compound of Formula (I) for use in neuroprotection or for use in the treatment of motoneuron degeneration.

In a particular embodiment, the present invention relates to a compound of Formula (I) for use in neuroprotection.

In a particular embodiment, the present invention relates to a compound of Formula (I) for use in the treatment of motoneuron degeneration.

In a particular embodiment, the present invention relates to a compound of Formula (I) for use in the treatment of ALS.

In a particular embodiment, the present invention relates to a compound of Formula (I) for use in preventing motoneuron death.

In a particular embodiment, the present invention relates to a compound of Formula (I) for use in preventing motoneuron death under chronic excitotoxic stress.

In a particular embodiment, the present invention relates to a compound of Formula (I) for use in enhancing motoneuron survival.

In a particular embodiment, the present invention relates to a compound of Formula (I) for use in enhancing motoneuron survival after rhizotomy.

In a particular embodiment, the present invention relates to a compound of Formula (I) for use in slowing motoneuron degeneration.

In a particular embodiment, the present invention relates to a compound of Formula (I) for use in preserving neuromuscular junctions innervation.

In a particular embodiment, the present invention relates to a compound of Formula (I) for use in protecting spinal motoneurons.

In a particular embodiment, the present invention relates to a compound of Formula (I) for use in reducing astroglial activation.

In a further aspect, the present invention relates to a pharmaceutical composition which comprises a compound of Formula (I) or a pharmaceutically acceptable salt thereof, and a pharmaceutically acceptable carrier, adjuvant or vehicle for use in neuroprotection or in the treatment of motoneuron degeneration.

In a further aspect, the present invention relates to a pharmaceutical composition which comprises a compound of Formula (I) or a pharmaceutically acceptable salt thereof, and a pharmaceutically acceptable carrier, adjuvant or vehicle for use in neuroprotection.

In a further aspect, the present invention relates to a pharmaceutical composition which comprises a compound of Formula (I) or a pharmaceutically acceptable salt thereof, and a pharmaceutically acceptable carrier, adjuvant or vehicle for use in the treatment of motoneuron degeneration.

Another aspect of the invention refers to a pharmaceutical composition which comprises a compound according to the invention as described above according to general formula I or a pharmaceutically acceptable salt or stereoisomer thereof, and a pharmaceutically acceptable carrier, adjuvant or vehicle. The present invention thus provides pharmaceutical compositions comprising a compound of this invention, or a pharmaceutically acceptable salt or stereoisomers thereof together with a pharmaceutically acceptable carrier, adjuvant, or vehicle, for administration to a patient.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts.

Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.05 to 1000 mg/kg/day.

The Compound of Formula (I) for use according to the invention, can be administered at a dosis of:
0.05 to 1000 mg/kg/day,
0.05 to 100 mg/kg/day,
0.05 to 50 mg/kg/day,
0.05 to 25 mg/kg/day,
0.05 to 10 mg/kg/day,
0.05 to 5 mg/kg/day, or
0.5 to 5 mg/kg/day.

The compounds and compositions of this invention may be used with other drugs to provide a combination therapy. The other drugs may form part of the same composition, or be provided as a separate composition for administration at the same time or at different time.

Another aspect of the invention refers to the use of a compound of the invention or a pharmaceutically acceptable salt or isomer thereof in the manufacture of a medicament.

Another aspect of the invention refers to a compound of the invention according as described above according to general formula I, or a pharmaceutically acceptable salt or isomer thereof, for use as a medicament in neuroprotection or for the treatment of motoneuron degeneration.

Another aspect of the invention refers to the use of a compound of the invention in the manufacture of a medicament for neuroprotection or for the treatment or prophylaxis of motoneuron degeneration.

Another aspect of this invention relates to neuroprotection or to a method of treating or preventing motoneuron degeneration which method comprises administering to a patient in need of such a treatment a therapeutically effective amount of a compound as above defined or a pharmaceutical composition thereof.

The present invention is illustrated below with the aid of examples. These illustrations are given solely by way of example and do not limit the general spirit of the present invention.

### General Experimental Part (Methods and Equipment of the Synthesis and Analysis

### Spinal Cord Organotypic Cultures (SCOCs)

SCOCs from lumbar sections of 8 day-old Sprague-Dawley rats were prepared as previously described (Mòdol-Caballero et al., 2017). Briefly, the spinal cord was collected and cut in 350 μm thick transverse sections with a Mcllwain Tissue Chopper. Four lumbar sections per sample were transferred onto Millicell-CM nets (0.4 μm, PICM03050, Millipore) in medium [50% (v/v) minimal essential medium (MEM, M5775, Sigma), 2 mM glutamine, 25 mM HEPES, 25% (v/v) Hank's Balanced Salt Solution (HBSS^{-/-}, 14175, Gibco) supplemented with 25.6 mg/ml glucose and 25% (v/v) heat-inactivated horse serum (26050-088, Gibco), pH=7.2]. Cultures were maintained at 37° C. in a 5% CO₂ humidified cabin and let to stabilize. After 7 days in vitro (DIV), DL-threo-b-hydroxyaspartic acid (THA; 100 μM) was added to induce chronic excitotoxicity (Rothstein et al., 1993). The neuroprotective effect of each Sig-1R ligand was assessed by its coaddition to the culture with THA and renewing at each medium change. Compound A (in DMSO 100 mM) and Compound B (in PBS 1 mM) were tested at four concentrations (30, 3, 0.3 and 0.03 μM). Comparisons were performed against slices with vehicle as negative control, and with addition of Riluzole (5 μM) as positive control (Guzmán-Lenis et al., 2009). In vitro experiments were performed in three independent cultures, with at least 12 different SCOCs for each experimental condition.

Slices were maintained for 28 DIV and then fixed with 4% paraformaldehyde. Fixed SCOC slices were immunolabeled with primary antibody mouse anti-neurofilament H non-phosphorylated (SMI-32, 1:500, BioLegend), washed and incubated with secondary antibody Alexa Fluor 488 donkey anti-mouse (1:500) (Mòdol-Caballero et al., 2017). Cell nuclei were labeled with DAPI (1:2000) and the sections mounted with Fluoromount-G medium (SouthernBiotech). Images of the ventral horn were captured with a confocal microscope (LSM 700 Axio Observer, Carl Zeiss 20×/z0.5). MN survival was assessed by counting all SMI-32 positive neurons in each spinal cord using the Cell Counter plugin of ImageJ software.

### Animals

For spinal nerve injury studies, adult female mice with B6SJL background were used, whereas for the ALS murine model, transgenic male mice carrying the G93A human mutation in SOD1 gene (B6SJL-Tg[SOD1-G93A]1Gur) and non-transgenic wild type (WT) littermates as controls were used (Mancuso et al., 2012b). The transgenic offspring was identified by polymerase chain reaction (PCR) amplification of DNA extracted from the tail. Mice were kept under standard conditions and handled in accordance with the guidelines of the European Union Council (Directive 2010/63/EU) and Spanish regulations on the use of laboratory animals. All experimental procedures were approved by the Ethics Committee of the Universitat Autònoma de Barcelona.

### Rhizotomy Procedure

Surgeries were performed in 3 months old mice under anesthesia with ketamine-xylazine (100-10 mg/kg i.p.) as previously described (Gaja-Capdevila et al., 2021). Briefly, the L4-L5 spinal roots were exposed by a small laminectomy on the right side. Then, roots were cut at the exit from the intervertebral foramina and the root stumps were separated. Mice were cared until recovery in warm environment. Analgesia was provided with buprenorphine (0.1 mg/kg) for the next 48 h post-surgery.

### Drug Administration

The following selective Sig-1R ligands were used: PRE-084 at a dose of 0.25 mg/Kg (TOCRIS), compound A and compound B at doses of 0.5 and 5 mg/kg (synthesized and supplied by Esteve Pharmaceuticals). The compounds were dissolved in 0.5% hydroxypropyl-methylcellulose (HPMC; Sigma-Aldrich) in distilled water. The compounds were administered by intraperitoneal (i.p.) route twice daily (bid) in a volume of 10 ml/kg. Administrations were given from 30 min after rhizotomy surgery until 42 days post-injury (dpi), the end of the study; in the SOD1^{G93A} study, treatments were given from 8 to 16 weeks of age.

For the spinal nerve injury (rhizotomy, rhizo), female WT mice were distributed in the following experimental groups: uninjured control (CTL) (n=16), rhizo+vehicle 0.5% HPMC (n=14), rhizo+PRE-084 0.25 mg/kg (n=4), rhizo+compound A 0.5 mg/kg (n=5), rhizo+compound A 5 mg/kg (n=6), rhizo+compound B 0.5 mg/kg (n=4), rhizo+compound B 5 mg/kg (n=5). For the SOD1^{G93A} studies, male transgenic mice were divided in 4 groups: SOD1+vehicle 0.5% HPMC (n=13), SOD1+PRE-084 0.25 mg/kg (n=5), SOD1+compound A 5 mg/kg (n=7), SOD1+compound B 5 mg/kg (n=6), with B6SJL male WT age-matched mice used as negative control of the disease (n=13).

### Pharmacokinetic Studies of Compound a and Compound B in Plasma and Brain

Adult female C57-BL6 mice (Charles River) were given a single intraperitoneal dose of 10 mg/kg of compound A or compound B in 0.5% HPMC (10 ml/kg). Blood and brain samples were extracted at selected time points (15 min, 1, 3 and 6 h) from two animals per sampling point. Blood samples were collected by intracardiac puncture into heparinized tubes. Plasma was obtained by blood centrifugation at 4° C. and 2280 g for 10 min. Plasma and brain samples were kept at −80° C. until analysis. Brains were homogenized with Dulbecco's phosphate saline buffer pH 7.4 (4 ml/g tissue). Plasma and brain samples were assayed by ultra-performance liquid chromatography-triple quadrupole mass spectrometry (UPLC-MS/MS) after protein precipitation. Standard pharmacokinetic parameters, such as the area under the curve (AUC), peak plasma concentration (Cₘₐₓ), time to peak concentration (tₘₐₓ), and terminal half-life (t_{1/2}) were determined by noncompartmental analysis of the plasma and brain concentration-time curves (Phoenix v.6.2.1.51, Pharsight, CA).

Plasma Levels Associated with Pharmacological Activity

Plasma levels were determined in the two in vivo models evaluated to be able to associate the pharmacological activity with the levels present in plasma. In the rhizotomy model, the plasma levels were evaluated at day 42 after surgery and at 15 min after compound administration (0.5 and 5 mg/kg i.p.). In the SOD1^{G93A} model, the plasma levels were evaluated at 16 weeks of age (after 8 weeks of administration) and 15 min after compound administration (5 mg/kg i.p.). Plasma samples were assayed by ultra-performance liquid chromatography-triple quadrupole mass spectrometry (UPLC-MS/MS) after plasma protein precipitation. Results are expressed as mean±standard deviation (n=2-6).

### Electrophysiological Tests

Motor nerve conduction tests were performed before the surgery to obtain baseline values and at the end of the follow up in the rhizotomy model, whereas the SOD1^{G93A} mice were evaluated at 8 weeks (prior to starting drug administration) and then every 2-3 weeks until the end point at 16 weeks of age. The sciatic nerve was stimulated by means of single pulses delivered through needle electrodes placed at the sciatic notch. The compound muscle action potential (CMAP) was recorded from tibialis anterior (TA), gastrocnemius (GM) and plantar interossei muscles with microneedle electrodes (Mancuso et al., 2011). The CMAPs were amplified to measure the latency to the onset and the amplitude from baseline to the maximal negative peak. The mice were anesthetized with pentobarbital (50 mg/kg i.p.) and their body temperature was maintained by means of a thermostated warming pad.

### Locomotion Tests

Rotarod test was performed to evaluate motor coordination and strength. The time that each animal remained on the rotating rod at a speed of 14 rpm was measured for five trials per mouse, and the longest time until falling was recorded; 180 sec was chosen as the cut-off time. The test was performed weekly from 8 to 16 weeks of age in SOD1^{G93A} and WT mice, and disease onset was determined as the first week when each mouse was unable to keep walking for 180 sec on the rod.

### Histological and Immunofluorescence Analyses

At the end of follow up (42 days post-injury (dpi)) for rhizotomy and 16 weeks of age for ALS mice) the animals were deeply anesthetized and transcardially perfused with 4% paraformaldehyde in PBS. The lumbar spinal cord and the TA muscle were harvested and cryopreserved in 30% sucrose solution in PB. The spinal cords were serially cut in 20 μm-thick transverse sections with a cryostat (Leica). For MN counting, L4-L5 spinal cord sections separated 100 μm were stained for 3 h with an acidified solution of 3.1 mM cresyl violet. MNs were identified by their localization in the lateral ventral horn of the spinal cord and were counted following strict size and morphological criteria: only MNs with diameter larger than 20 μm, polygonal shape and prominent nucleoli were counted (Gaja-Capdevila et al., 2021; Mancuso et al., 2012a).

For glial cells immunofluorescence, other lumbar spinal cord sections were blocked with 10% normal donkey serum and incubated overnight with primary antibodies rabbit anti-Iba1 (1:500; 019-19,741, Wako) and rat anti-GFAP (1:500; 13-0300, Invitrogen) to label microglia and astroglia, respectively. After several washes, sections were incubated with secondary antibodies Alexa Fluor 488-donkey anti-rat (1:500) or Alexa Fluor 594 donkey anti-rabbit (1:500). Finally, sides were mounted with Fluoromount G. To quantify glial cell reactivity, images of the ventral horn were acquired with an epifluorescence microscope (Nikon Eclipse Ni, Japan) using the same conditions for each analyzed marker. After defining a threshold for background correction, the integrated density of GFAP or Iba1 labeling was measured using ImageJ software.

For neuromuscular junctions (NMJ) labeling, 50 μm-thick longitudinal sections of TA muscle were serially cut. Sections were blocked with 5% normal donkey serum and incubated with primary antibodies rabbit anti-synaptophysin (1:500; AB32127, Abcam) and chicken anti-neurofilament 200 (NF200, 1:1000; AB5539, Millipore, USA) 48 h at 4° C. After washes, sections were incubated overnight with secondary antibody Alexa Fluor 594-donkey anti-rabbit and anti-chicken (1:200; A11042-A21207, Invitrogen, USA) and Alexa 488 conjugated α-bungarotoxin (1:500; B13422, Life Technologies, USA). Confocal images were captured (LSM 700 Axio Observer, Carl Zeiss, 40×Oil/z0.5) and the maximum projection images generated from 1.3 μm z projections. The proportion of innervated NMJs was calculated by classifying each endplate as occupied (when presynaptic terminals overly the endplate) or vacant (no presynaptic label in contact with the endplate). At least 60 endplates were analyzed per muscle.

### Statistical Analysis

All data are expressed as mean±standard error of the mean (SEM). Statistics were performed using GraphPad Prism 6 software. Histological data were analyzed using One-way ANOVA followed by Bonferroni post hoc test for multiple comparisons, and for in vitro pharmacological profile Dunnett's post-hoc multiple comparisons was used. Electrophysiological and functional measurements were statistically analyzed using One-way or Two-way ANOVA followed by Bonferroni post-hoc test for multiple comparisons. Statistical significance was set at p<0.05.

### Results

### Example 1: In Vitro Pharmacological Profile of Compound a and Compound B

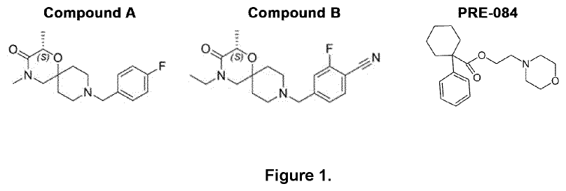

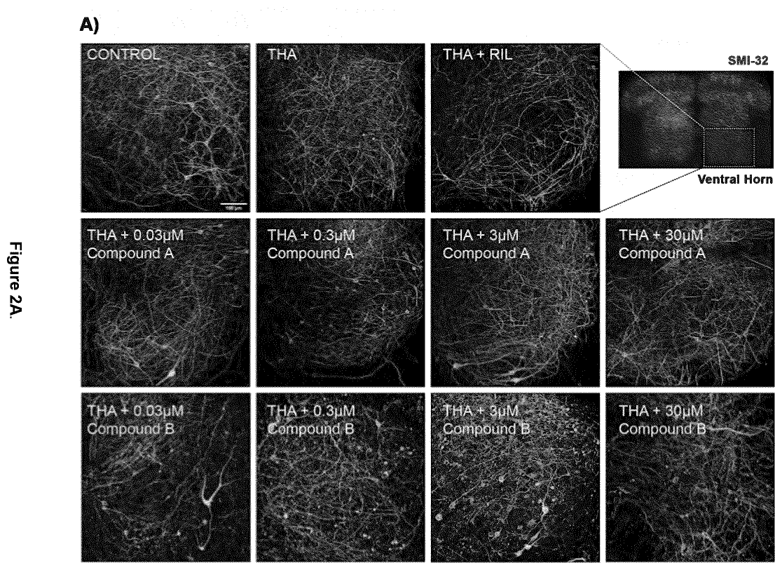

The two compounds synthesized, compound A and compound B (FIG. 1), were highly selective Sig-1R ligands (WO 2017084752). They showed high affinity for the human Sig-1R while they did not show affinity for the Sig-2R (IC₅₀>10000 nM for both of them, not shown). A selectivity profile (Spectrum Screen) including a panel with more than 180 binding assays for different receptors, ion channels, transporters and enzymes was performed for compound A and compound B at EurofinsPanlabs according to their specifications. Both compounds showed no significant affinity (% inhibition<50% at 10 μM) for any of the targets included in the selectivity panel, apart from the Sig-1R. Similarly, PRE-084 has good affinity for the human Sig-1R and no affinity for human Sig-2R (IC₅₀>10000 nM) (not shown).

### Example 2: MN Death in SCOC Under Chronic Excitotoxic Stress

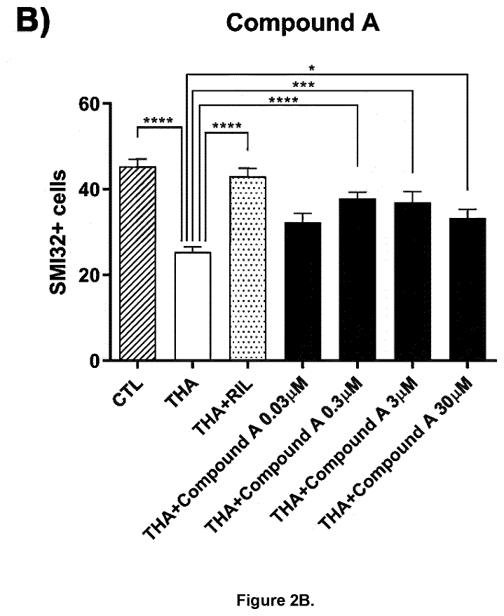

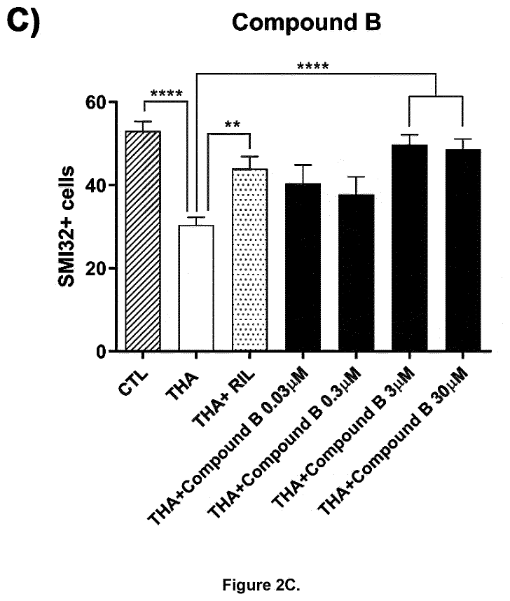

In the SCOC, the number of SMI-32 positive MNs was significantly reduced in the ventral horn of slices treated with THA compared to the control slices (FIG. 2). Riluzole, used as a positive control against excitotoxicity, preserved MNs at levels of control cultures. As shown in FIG. 2B, slices treated with compound A had significantly more SMI-32 positive neurons at three concentrations tested with the highest effect at 0.3 to 3 μM (mean±SEM; n=20-24 hemisections per treatment). The two highest concentrations of compound B assessed (3 and 30 μM) also significantly preserved MNs (FIG. 2C). One-way ANOVA followed by Bonferroni's post hoc test showed significant differences noted in FIG. 2C; ****p<0.0001, ***p<0.001; **p<0.01, *p<0.05 vs THA condition.

Therefore, Compound A and Compound B prevent MN death in SCOC under chronic excitotoxic stress.

### Example 3: Pharmacokinetic Analyses of Sig-1R Ligands in Plasma and Brain

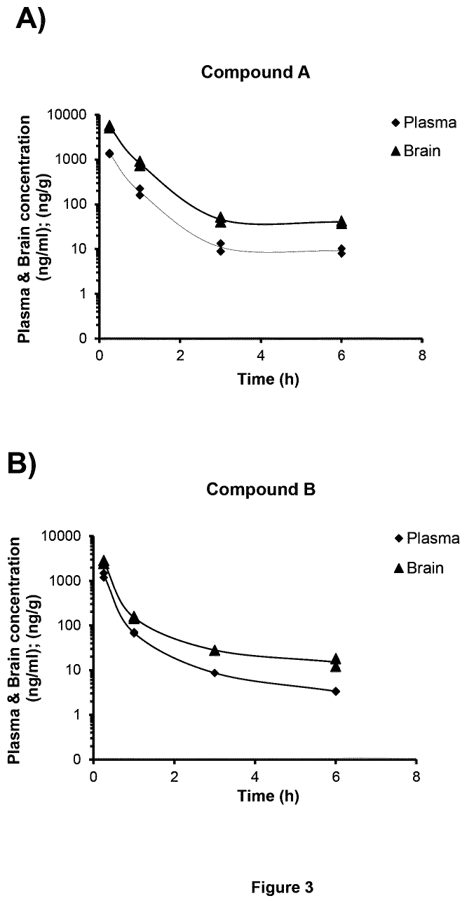

The pharmacokinetic profile of Sig-1R ligands compound A and compound B was determined in female mice after intraperitoneal administration of a single dose of 10 mg/kg (Table 1, FIG. 3). At 15 min, 1, 3 and 6 h samples of blood and brain were extracted to determine the concentrations of both compounds (n=2 mice per sampling point). Curves are shown in semilogarithmic scale. Brain and plasma kinetics were parallel for both compounds. Brain concentrations were higher than the corresponding plasma concentrations as shown by a brain-to-plasma ([brain]/[plasma]) ratio based on AUC of 4.4 and 2.1 for compound A and compound B, respectively. These results indicate that these compounds have a good penetration into the brain, necessary to act in the central nervous system (CNS). PRE-084 is also known to penetrate the brain ([brain]/[plasma] ratio about 1.2 at Tmax (5 min) following single i.p. administration at 10 mg/kg to male CD1 mice) (Marra et al., 2016).

### Example 4: MN Survival after Rhizotomy

After rhizotomy surgery, nerve conduction tests were done to confirm the complete denervation of both TA and GM muscles, thus ensuring a complete L4 and L5 root section. There were no recordable CMAPs from TA and GM muscles, and only partial preservation of the plantar muscle CMAP in all the mice included in the study after surgery and at the end of the follow-up, as in previous studies (Gaja-Capdevila et al., 2021).

In the rhizotomy mice, a similar plasma concentration, about 20 ng/ml, was found for compound A and compound B 15 min after the last 0.5 mg/kg i.p. administration (day 42 after surgery, end of the study). At 5 mg/kg i.p. the levels obtained in plasma for compound A were lower than those obtained for compound B, 265 and 592 ng/ml, respectively (Table 2). However, taking into account the brain-to-plasma ratio (4.4 for compound A and 2.1 for compound B) (Table 1), estimated brain concentrations at 5 mg/kg i.p. are similar for both compounds.

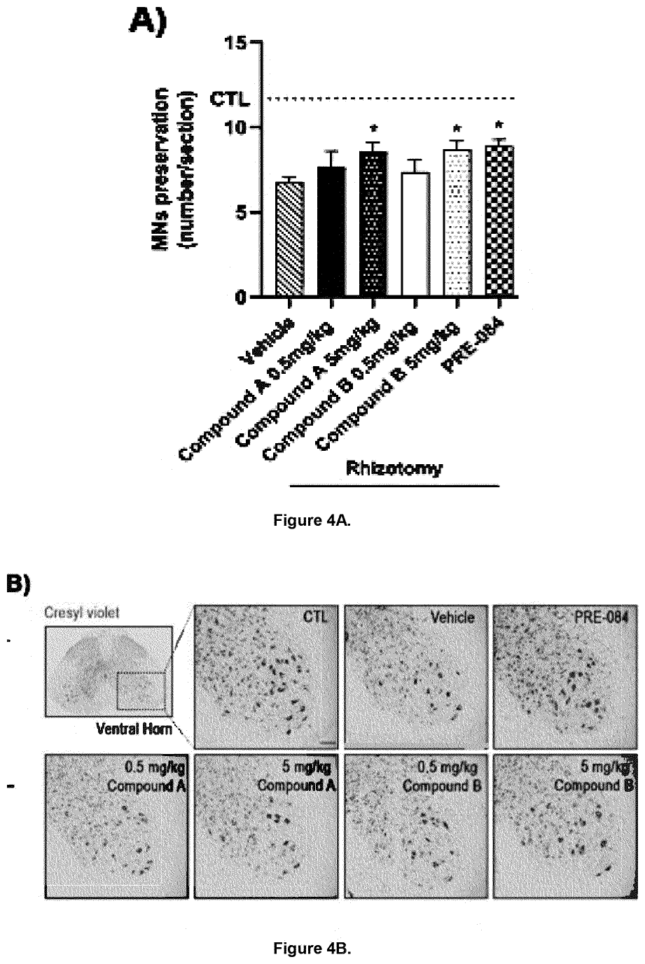

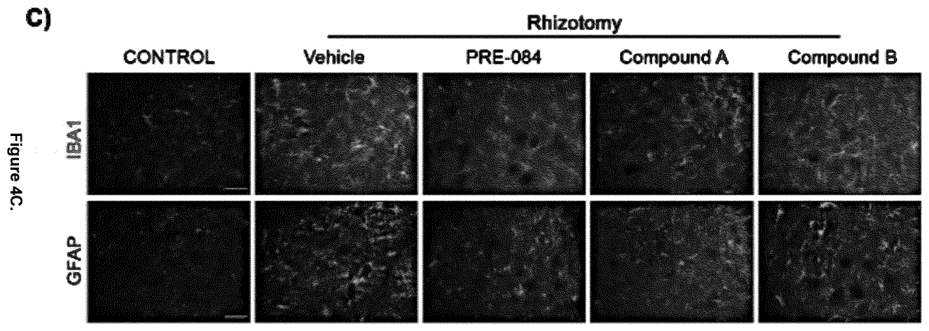

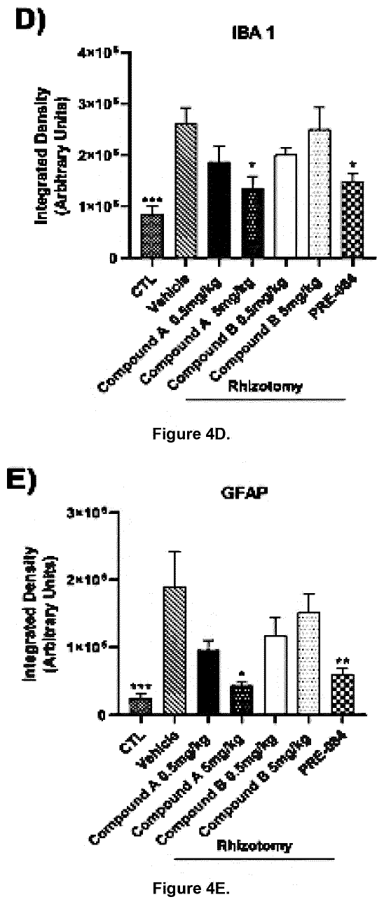

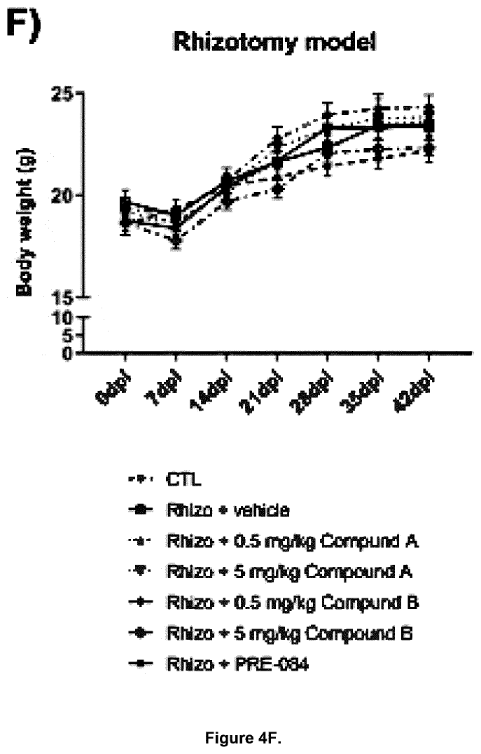

Histological analyses revealed that rhizotomy (L4-L5 spinal nerve section) caused severe MN loss (43%) in the affected lumbar segments and that the three Sig-1R ligands (compound A, compound B, and PRE-084 as positive control) reduced MN degeneration at 42 days after the injury. Rhizotomized mice treated during 6 weeks with compound A and compound B at a dose of 5 mg/kg/bid i.p. had a significantly higher number of spinal MNs (8.6±0.6 and 8.7 0.5, respectively) compared to the untreated injured group (6.8±0.3) in the ipsilateral ventral horn of spinal cord sections stained with cresyl violet (FIG. 4A, Plot of number of α-MNs in L4-L5 segments, showing higher number of MNs in mice treated with Sig-1R ligands than in untreated ones. Animals per group: control n=14, vehicle n=14, PRE-084 n=4, compound A 0.5 mg/kg n=5 and 5 mg/kg n=6, compound B 0.5 mg/kg n=4 and 5 mg/kg n=5, and FIG. 4B, Representative histological images). The administration of compound A and compound B Sig-1R ligands increased the number of surviving MNs at the same level as PRE-084 (8.9±0.4), used as a positive control of MN preservation (Gaja-Capdevila et al., 2021; Mancuso et al., 2012a; Penas et al., 2011). Administration of compound A and compound B at the low dose (0.5 mg/kg) did not exert significant MN protective effects (7.6±0.9 and 7.4±0.7, respectively) after rhizotomy.

As a consequence of the rhizotomy, microglia and astrocytes were activated surrounding lumbar MNs. IBA1 and GFAP immunostaining showed a marked increase in the untreated rhizotomized group (26×10⁴±3×10⁴ and 19×10⁴±5×10⁴, respectively; mean of integrated density±SEM) compared with control mice (8×10⁴±1.5×10⁴ and 2.5×10⁴±0.6×10⁴) (FIG. 4C, PRE-084, compound A and compound B at dose of 5 mg/kg) at 42 dpi. Scale bar 50 μm; and FIGS. 4D-E, wherein n=3-6 mice per group). Regarding pharmacologic treatments, compound A at the highest dose significantly diminished microglial (13×10⁴±2×10⁴) and astroglial (4×10⁴±0.4×10⁴) reactivity at levels similar to the positive control PRE-084 (15×10⁴±1.4×10⁴ and 6×10⁴±0.9×10⁴). In contrast, compound B at 5 mg/kg did not significantly reduce glial reactivity (25×10⁴±4×10⁴ and 15×10⁴±2.7×10⁴). The low doses tested of both Sig-1R ligands showed a non-significant tendency to decrease microgliosis and astrogliosis.

Finally, it is important to note that twice daily administration for 6 weeks of Compounds A and B did not result in noticeable side effects on mice, with no difference in body weight compared with the vehicle group, thus indicating that chronic administration of compound A and compound B did not cause toxicity in the animals (FIG. 4F, All the mice groups presented a mild reduction of the body weight the first week after surgery, then gained weight normally during the follow-up. Data are mean±SEM, analyzed with One-way (A,D,E) or Two-way (F) ANOVA and Bonferroni's multiple comparisons test. *p<0.05, **p<0.001,***p<0.0001 vs vehicle rhizotomized mice). No abnormalities in motor control and behavior were observed.

Therefore, Compound A and compound B enhance MN survival after rhizotomy.

### Example 5: Disease Progression in SOD1^{G93A} Mice

The neuroprotective effect of the Sig-1R ligands Compound A and Compound B were also assessed in the SOD1^{G93A} mice model of ALS. In this study only the dose of 5 mg/kg of each compound was tested based on previous results in the rhizotomy model. After administration of 5 mg/kg i.p. for 8 weeks (from 8 to 16 weeks), plasma concentrations 15 min after last dose administration were around 500 ng/ml, similar for both Sig-1R ligands, compound A and compound B (Table 2). Considering the brain-to-plasma ratio of compounds (4.4 for compound A and 2.1 for compound B see Table 1), the estimated brain concentration of compound A is about two-fold the concentration of compound B.

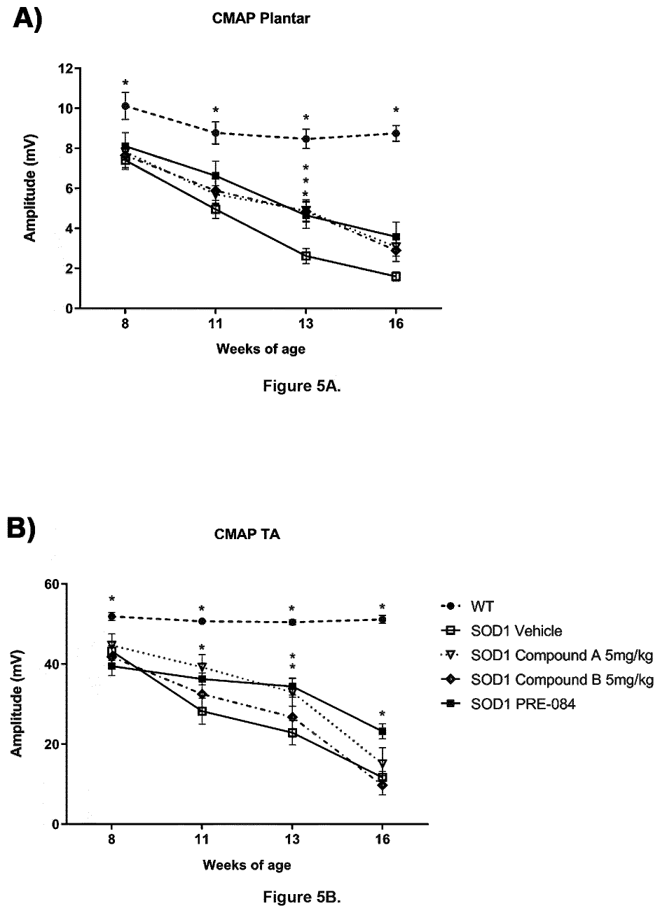

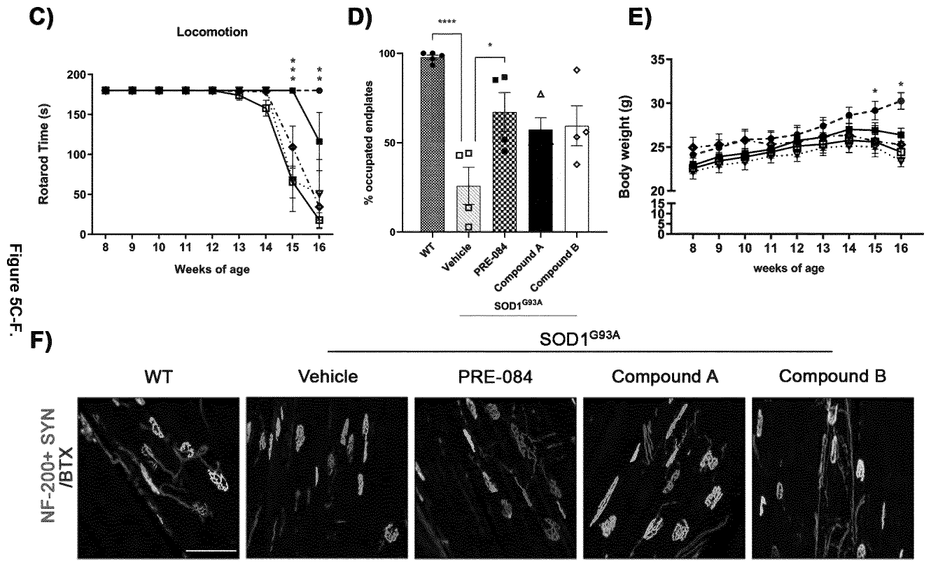

At early (11 weeks) and mid (13 weeks) stages of the disease, motor nerve conduction tests showed that administration of compound A significantly prevented the decline in amplitude of the CMAP of TA and plantar muscles, similar to PRE-084 (FIG. 5A-B). The histological analyses of NMJ of the hindlimb muscle showed that PRE-084 treatment significantly increased the proportion of innervated endplates compared to untreated SOD1^{G93A} mice at 16 weeks, consistent with the electrophysiological results of higher CMAP amplitude values. Compound A and compound B caused a tendency to preserve NMJ innervation,) (FIG. 5D-F, Data are mean±SEM, analyzed with One-way (D) or Two-way (A, B, C, E) ANOVA with Bonferroni's multiple comparisons test. ****p<0.0001,*p<0.05 vs. SOD1^{G93A} vehicle).

Similar to findings in the rhizotomy study, the daily intraperitoneal administration of the Sig-1R ligands compound A, compound B and PRE-084 to SOD1^{G93A} mice did not cause differences in body weight compared with the vehicle group (FIG. 5E).

Therefore, Compound A treatment slows disease progression in SOD1^{G93A} mice.

### Example 6: Spinal MNs and Astroglial Activation in SOD1^{G93A} Mice

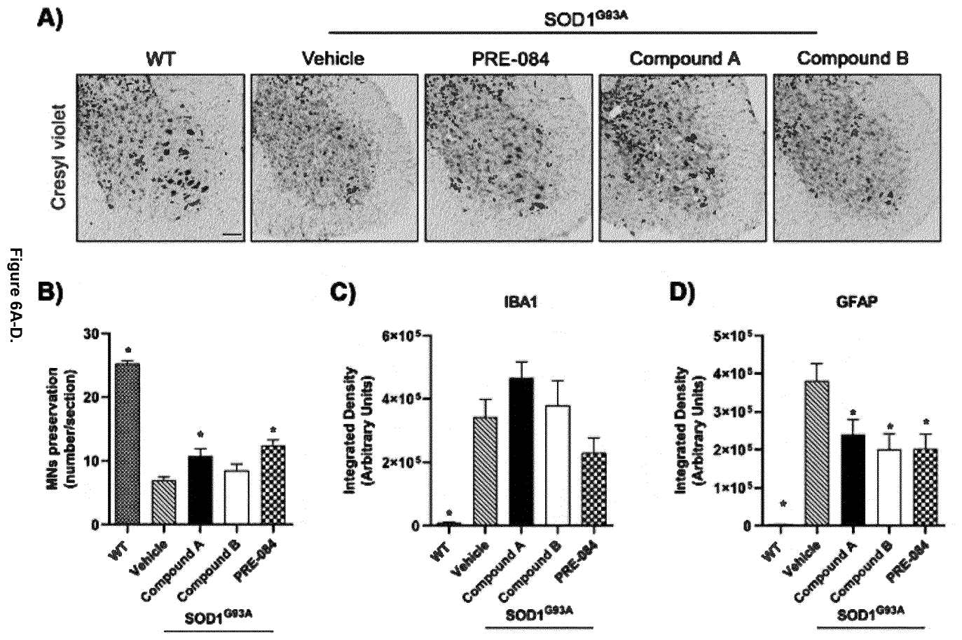

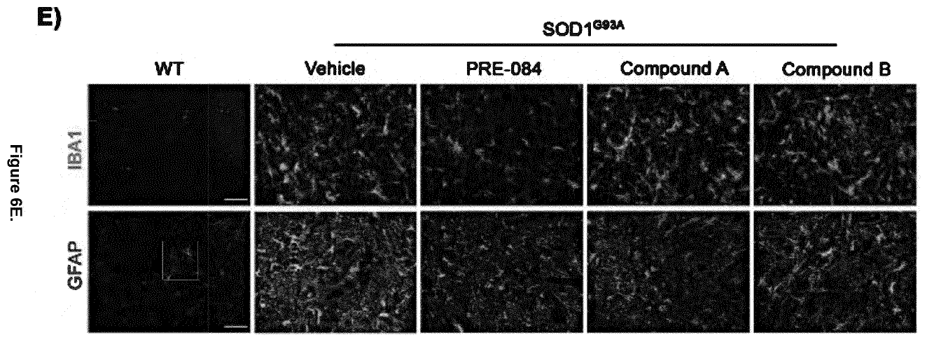

Counts of α-MNs in ventral horns of spinal cord sections revealed that untreated SOD1^{G93A} mice had a loss of around 60% MNs (6.9±0.5 MNs per section) compared to WT mice (25.3±0.5) at 16 weeks of age. SOD1^{G93A} mice treated with compound A (5 mg/kg) had a significantly higher number of surviving spinal MNs (10.8±1.1), like animals administered with PRE-084 (12.4±0.9), whereas compound B produced a less pronounced effect (8.5±1.0) (FIG. 6A, and FIG. 6B, Animals per group WT n=13, vehicle n=16, PRE-084 n=5, compound A n=7, compound B n=5). Glial reactivity showed a huge increase in SOD1^{G93A} with respect to WT mice. Treatment with the new Sig-1R ligands compound A and compound B produced a significant decrease of astrocyte activation as did PRE-084, (FIG. 6C-E, n=5-10 mice per group. Data are expressed as mean±SEM, and analyzed by One-way ANOVA and Bonferroni's multiple comparisons test. *p<0.05 vs SOD1^{G93A} vehicle mice).

Therefore, Compound A treatment protects spinal MNs and reduces astroglial activation in SOD1^{G93A} mice.

### CONCLUSIONS

The pharmacological activity and neuroprotective effect on MN degeneration of two potent and selective Sig-1R ligands, named Compound A and Compound B, is reported. In vitro treatment with compound A and compound B significantly reduced MN death caused by chronic excitotoxicity. In vivo, Sig-1R ligand compound A had more potent effect on preventing MN degeneration than compound B. In addition, administration of compound A had effects on reducing astroglial reactivity in both models, and compound B only in the SOD1^{G93A} mice model. The observation that Sig-1R ligands exert protective/preventive effects on different experimental models of MN degeneration opens promising perspectives for targeting Sig-1R for MND.

### Tables:

TABLE 1
Pharmacokinetic parameters of compound A and compound B
compounds and PRE-084 in plasma and brain after single
intraperitoneal administration of 10 mg/kg to female mice.

| | | | Cₘₐₓ | | AUC | |
|---|---|---|---|---|---|---|
| | Sample | t_{1/2} | (ng/ml | tₘₐₓ | (ng · h/ml or | Brain-to-plasma |
| Compound | type | (h) | or ng/g) | (h) | ng · h/g) | AUC ratio |
| Compound A | Plasma | >5 | 1348 | 0.25 | 1116 | 4.4 |
| | Brain | >5 | 5405 | 0.25 | 4873 | |
| Compound B | Plasma | 2.2 | 1343 | 0.25 | 802 | 2.1 |
| | Brain | 3.4 | 2652 | 0.25 | 1698 | |
| PRE-084^{a,b} | Plasma | — | 659 | — | — | 1.2 |
| | Brain | — | 774 | — | — | |

t_{1/2}: terminal half-life,
Cₘₐₓ: peak plasma concentration,
tₘₐₓ: time to peak concentration,
AUC: area under the curve.
^{a}From literature (Marra et al., 2016).
^{b}Concentrations determined at 5 min post-administration.

TABLE 2
Plasma levels of compound A, compound B and PRE-
084 after chronic intraperitoneal administration.

| | | | Plasma concentration (ng/ml) | |
|---|---|---|---|---|
| | | Dose | 15 min post-administration | |
| Model | Compound | (mg/kg) | Mean | SD |
| Spinal Nerve | Compound A | 0.5 | 20 | 8 |
| Injury female | | 5 | 265 | 53 |
| mouse | Compound B | 0.5 | 21 | 7 |
| | | 5 | 592 | 176 |
| | PRE-084^{a} | 0.25 | 3.6 | 0.8 |
| Transgenic | Compound A | 5 | 425 | 129 |
| SOD1^{G93A} | Compound B | 5 | 692 | 206 |
| male mouse | PRE-084^{a} | 0.25 | 2.6 | 1.3 |

^{a}Concentrations determined at 30 min post-administration

### REFERENCES

Al-Saif, A., Al-Mohanna, F., Bohlega, S., 2011. Ann. Neurol. 70, 913-919.
Edaravone (MCI-186) ALS 19 Study Group, 2017. Lancet Neurol. 16, 505-512.
Gaja-Capdevila, N., Hernández, N., Zamanillo, D., Vela, J. M., Merlos, M., Navarro, X., Herrando-Grabulosa, M., 2021. Int. J. Mol. Sci. 22, 6956.
Guzmán-Lenis, M.-S., Navarro, X., Casas, C., 2009. Neuroscience 162, 31-8.
Ionescu, A., Gradus, T., Altman, T., Maimon, R., Saraf Avraham, N., Geva, M., Hayden, M., Perlson, E., 2019. Cell Death & Disease volume 10, Article number: 210 (2019)
Langa, F., Codony, X., Tovar, V., Lavado, A., Gimenez, E., Cozar, P., Cantero, M., Dordal, A., Hernández, E., Perez, R., Monroy, X., Zamanillo, D., Guitart, X., Montoliu, L., 2003. Eur. J. Neurosci. 18, 2188-2196.
Ludolph, A. C., Jesse, S., 2009. Ther. Adv. Neurol. Disord. 2(5): 319-326
Luty, A. A., Kwok, J. B. J., Dobson-Stone, C., Loy, C. T., Coupland, K. G., Karlström, H., Sobow, T., Tchorzewska, J., Maruszak, A., Barcikowska, M., Panegyres, P. K., Zekanowski, C., Brooks, W. S., Williams, K. L., Blair, I. P., Mather, K. A., Sachdev, P. S., Halliday, G. M., Schofield, P. R., 2010. Ann. Neurol. 68, 639-649.
Mancuso, R., Navarro, X., 2015. Prog. Neurobiol. 133, 1-26.
Mancuso, R., Oliván, S., Mancera, P., Pastén-Zamorano, A., Manzano, R., Casas, C., Osta, R., Navarro, X., 2012a. Amyotroph. Lateral Scler. 13, 302-10.
Mancuso, R., Oliván, S., Rando, A., Casas, C., Osta, R., Navarro, X., 2012b. Neurotherapeutics volume 9, pages 814-826 (2012)
Mancuso, R., Santos-Nogueira, E., Osta, R., Navarro, X., 2011. Clin. Neurophysiol. 122, 1660-70
Marra, A., Rossi, D., Pignataro, L., Bigogno, C., Canta, A., Oggioni, N., Malacrida, A., Corbo, M., Cavaletti, G., Peviani, M., Curti, D., Dondio, G., Collina, S., 2016. Future Med. Chem. 8, 287-295.
Mòdol-Caballero, G., Santos, D., Navarro, X., Herrando-Grabulosa, M., 2017. Front. Cell. Neurosci. 11, 431.
Ono, Y., Tanaka, H., Takata, M., Nagahara, Y., Noda, Y., Tsuruma, K., Shimazawa, M., Hozumi, I., Hara, H., 2014. Neuroscience Letters. 559: 174-178
Penas, C., Pascual-Font, A., Mancuso, R., Forés, J., Casas, C., Navarro, X., 2011. J. Neurotrauma 28, 831-40
Peviani, M., Salvaneschi, E., Bontempi, L., Petese, A., Manzo, A., Rossi, D., Salmona, M., Collina, S., Bigini, P., Curti, D., 2014. Neurobiol. Dis. 62, 218-232.
Rothstein, J. D., Jin, L., Dykes-Hoberg, M., Kuncl, R. W., 1993. Proc. Natl. Acad. Sci. U.S.A 90, 6591-6595
Ververis, A., Dajani, R., Koutsou, P., Aloqaily, A., Nelson-Williams, C., Loring, E., Arafat, A., Mubaidin, A. F., Horany, K., Bader, M. B., Al-Baho, Y., Ali, B., Muhtaseb, A., Despenza, T., Al-Qudah, A. A., Middleton, L. T., Zamba-Papanicolaou, E., Lifton, R., Christodoulou, K., 2019. J Med Genet 2020; 57:178-186.

1-15. (canceled)

16. A method of providing neuroprotection and/or treating motoneuron degeneration in a subject in need thereof, comprising administration of a compound of Formula (I): 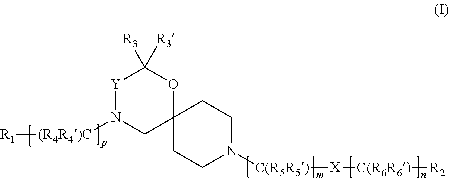
wherein
p is 0 or 1 or 2;
m is 1, 2 or 3;
n is 0, 1 or 2;
Y is —CH₂— or —C(O)—;
X is a bond, —C(RₓR_{x′})—, —C(O)— or —O—;
wherein Rₓ is selected from halogen, substituted or unsubstituted C₁₋₆ alkyl, substituted or unsubstituted C₂₋₆ alkenyl substituted or unsubstituted C₂₋₆ alkynyl, and —OR₈;
R_{x′} is selected from hydrogen, halogen or substituted or unsubstituted C₁₋₆ alkyl, substituted or unsubstituted C₂₋₆ alkenyl and substituted or unsubstituted C₂₋₆ alkynyl;
R₈ is selected from hydrogen, substituted or unsubstituted C₁₋₆ alkyl, substituted or unsubstituted C₂₋₆ alkenyl and substituted or unsubstituted C₂₋₆ alkynyl;
R₁ is selected from substituted or unsubstituted C₁₋₆ alkyl, substituted or unsubstituted C₂₋₆ alkenyl, substituted or unsubstituted C₂₋₆ alkynyl and substituted or unsubstituted cycloalkyl;
wherein the cycloalkyl in R, if substituted, is substituted with one or more substituent/s selected from halogen, —R₁₁, —OR₁₁, —NO₂, —NR₁₁R_{11′″}, NR₁₁C(O)R_{11′}, —NR₁₁S(O)₂R_{11′}, —S(O)₂NR₁₁R_{11′}, —NR₁₁C(O)NR_{11′}R_{11″}, —SR₁₁, —S(O)R₁₁, S(O)₂R₁₁, —CN, haloalkyl, haloalkoxy, —C(O)OR₁₁, —C(O)NR₁₁R_{11′}, —OCH₂CH₂OH, —NR₁₁S(O)₂NR_{11′}R_{11″} and C(CH₃)₂OR₁₁;
additionally, the cycloalkyl in R₁, if substituted, may also be substituted with 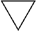
or ═O;
wherein the alkyl, alkenyl or alkynyl in R₁, if substituted, is substituted with one or more substituent/s selected from —OR₁₁, halogen, —CN, haloalkyl, haloalkoxy and —NR₁₁R_{11′″};
wherein R₁₁, R_{11′} and R_{11″} are independently selected from hydrogen, unsubstituted C₁₋₆ alkyl, unsubstituted C₂₋₆ alkenyl and unsubstituted C₂₋₆ alkynyl;
and wherein R₁˜ is selected from hydrogen, unsubstituted C₁₋₆ alkyl, unsubstituted C₂₋₆ alkenyl, unsubstituted C₂₋₆ alkynyl and -Boc;
R₂ is selected from hydrogen, substituted or unsubstituted C₁₋₆ alkyl, substituted or unsubstituted C₂₋₆ alkenyl, substituted or unsubstituted C₂₋₆ alkynyl, substituted or unsubstituted cycloalkyl, substituted or unsubstituted aryl and substituted or unsubstituted heterocyclyl,
wherein the cycloalkyl, aryl or heterocyclyl in R₂, if substituted, is substituted with one or more substituent/s selected from halogen, —R₁₂, —OR₁₂, —NO₂, —NR₁₂R_{12′″}, NR₁₂C(O)R_{12′}, —NR₁₂S(O)₂R_{12′}, —S(O)₂NR₁₂R_{12′}, —NR₁₂C(O)NR_{12′}R_{12″}, —SR₁₂, —S(O)R₁₂, S(O)₂R₁₂, —CN, haloalkyl, haloalkoxy, —C(O)OR₁₂, —C(O)NR₁₂R_{12′}, —OCH₂CH₂OH, —NR₁₂S(O)₂NR_{12′}R_{12″} and C(CH₃)₂OR₁₂;
additionally, the cycloalkyl or non-aromatic heterocyclyl in R₂, if substituted, may also be substituted with 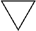
or ═O;
wherein the alkyl, alkenyl or alkynyl in R₂, if substituted, is substituted with one or more substituent/s selected from —OR₁₂, halogen, —CN, haloalkyl, haloalkoxy and —NR₁₂R_{12″″};
wherein R₁₂, R_{12′}, and R_{12′″} are independently selected from hydrogen, unsubstituted C₁₋₆ alkyl, unsubstituted C₂₋₆ alkenyl and unsubstituted C₂₋₆ alkynyl;
and wherein R₁₂ is selected from hydrogen, unsubstituted C₁₋₆ alkyl, unsubstituted C₂₋₆ alkenyl, unsubstituted C₂₋₆ alkynyl and -Boc;
R₃ and R_{3′} are independently selected from hydrogen, substituted or unsubstituted C₁₋₆ alkyl, substituted or unsubstituted C₂₋₆ alkenyl and substituted or unsubstituted C₂₋₆ alkynyl;
alternatively, R₃ and R₃, taken together with the connecting C-atom, form a substituted or unsubstituted cycloalkyl;
R₄ and R_{4′} are independently selected from hydrogen, substituted or unsubstituted C₁₋₉ alkyl, substituted or unsubstituted C₂₋₉ alkenyl, substituted or unsubstituted C₂₋₉ alkynyl, —CHOR₉ and —C(O)OR₉;
wherein R₉ is selected from hydrogen, substituted or unsubstituted C₁₋₉ alkyl, substituted or unsubstituted C₂₋₉ alkenyl and substituted or unsubstituted C₂₋₉ alkynyl;
R₅ and R_{5′} are independently selected from hydrogen, substituted or unsubstituted C₁₋₆ alkyl, substituted or unsubstituted C₂₋₆ alkenyl, substituted or unsubstituted C₂₋₆ alkynyl, —CHOR₇ and —C(O)OR₇;
wherein R₇ is selected from hydrogen, substituted or unsubstituted C₁₋₆ alkyl, substituted or unsubstituted C₂₋₆ alkenyl and substituted or unsubstituted C₂₋₆ alkynyl;
alternatively, R₅ and R_{5′}, taken together with the connecting C-atom, form a substituted or unsubstituted cycloalkyl or a substituted or unsubstituted non-aromatic heterocyclyl;
R₆ and R_{6′} are independently selected from hydrogen, substituted or unsubstituted C₁₋₆ alkyl, substituted or unsubstituted C₂₋₆ alkenyl, substituted or unsubstituted C₂₋₆ alkynyl, —OR₁₀, —CHOR₁₀ and —C(O)OR₁₀;
wherein R₁₀ is selected from hydrogen, substituted or unsubstituted C₁₋₆ alkyl, substituted or unsubstituted C₂₋₆ alkenyl and substituted or unsubstituted C₂₋₆ alkynyl;
optionally as a stereoisomers, including enantiomers and diastereomers, a racemate or a mixture of at least two stereoisomers, including enantiomers and/or diastereomers, in any mixing ratio, or a salt thereof, or a solvate thereof,
wherein the compound of Formula (I) is administered alone or in combination with one or more pharmaceutically acceptable excipients.

17. The method according to claim 16, wherein p is 0 or 1.

18. The method according to claim 16, wherein
R₃ is selected from substituted or unsubstituted C₁₋₆ alkyl, substituted or unsubstituted C₂₋₆ alkenyl and substituted or unsubstituted C₂₋₆ alkynyl;
R₃, is selected from hydrogen, substituted or unsubstituted C₁₋₆ alkyl, substituted or unsubstituted C₂₋₆ alkenyl and substituted or unsubstituted C₂₋₆ alkynyl;
alternatively, R₃ and R_{3′}, taken together with the connecting C-atom, form a substituted or unsubstituted cycloalkyl.

19. The method according to claim 18, wherein R₃ is substituted or unsubstituted C₁₋₆ alkyl.

20. The method according to claim 18, wherein R₃ is hydrogen.

21. The method according to claim 16, wherein the compound of Formula (I) is a compound of Formula (I′), (I^{a′}), (I^{b′}) or (I^{c′})

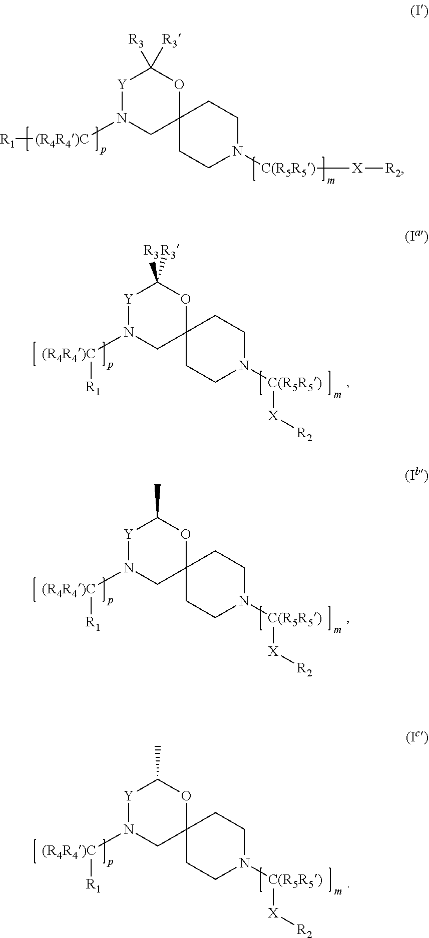

22. The method according to claim 16, wherein the compound of Formula (I) is a compound of Formula (I^{2′})

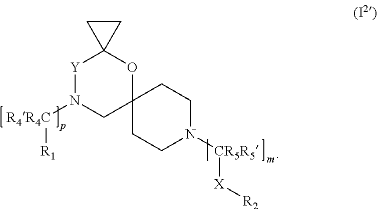

23. The method according to claim 16, wherein the compound of Formula (I) is a compound of Formula (I^{3′})

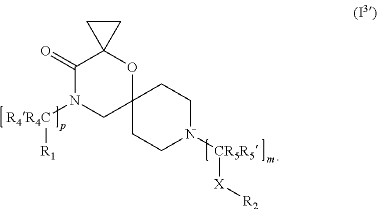

24. The method according to claim 16, wherein X is a bond or —O—.

25. The method according to claim 16, wherein the compound of Formula (I) is a compound of Formula (I^{4′})

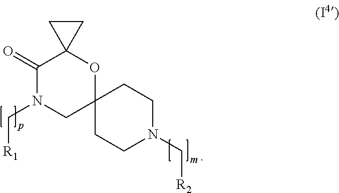

26. The method according to claim 16, wherein the compound of Formula (I) is a compound of Formula (I^{5′})

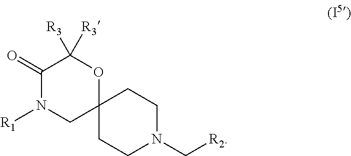

27. The method according to claim 16, wherein
R₁ is substituted or unsubstituted C₁₋₆ alkyl.

28. The method according to claim 27, wherein R₁ is a substituted or unsubstituted group selected from methyl, ethyl and propyl.

29. The method according to claim 16, wherein R₁ is selected from unsubstituted C₁₋₆ alkyl and unsubstituted cycloalkyl.

30. The method according to claim 29, wherein R₁ is an unsubstituted group selected from methyl, ethyl and propyl.

31. The method according to claim 16, wherein
R₂ is selected from hydrogen, substituted or unsubstituted C₁₋₆ alkyl, substituted or unsubstituted aryl and substituted or unsubstituted heterocyclyl.

32. The method according to claim 16, wherein R₂ is hydrogen or a substituted or unsubstituted group selected from methyl, ethyl, propyl, isopropyl, isobutyl, phenyl and pyridine.

33. The method according to claim 16, wherein R₂ is unsubstituted group selected from methyl, ethyl, propyl, isopropyl and isobutyl.

34. The method according to claim 16, wherein the compound of Formula (I) is selected from the group consisting of:
12-ethyl-8-isopentyl-4-oxa-8,12-diazadispiro[2.1.5.3]tridecan-13-one,
4-ethyl-9-isopentyl-1-oxa-4,9-diazaspiro[5.5]undecan-3-one,
8-isopentyl-12-methyl-4-oxa-8,12-diazadispiro[2.1.5.3]tridecan-13-one,
(R)-4-ethyl-9-isopentyl-2-methyl-1-oxa-4,9-diazaspiro[5.5]undecan-3-one,
8-isopentyl-12-isopropyl-4-oxa-8,12-diazadispiro[2.1.5.3]tridecan-13-one,
12-ethyl-8-isobutyl-4-oxa-8,12-diazadispiro[2.1.5.3]tridecan-13-one,
(S)-4-ethyl-9-isopentyl-2-methyl-1-oxa-4,9-diazaspiro[5.5]undecan-3-one,
12-ethyl-8-(2-isopropoxyethyl)-4-oxa-8,12-diazadispiro[2.1.5.3]tridecan-13-one,
8-isopentyl-12-propyl-4-oxa-8,12-diazadispiro[2.1.5.3]tridecan-13-one,
12-isobutyl-8-isopentyl-4-oxa-8,12-diazadispiro[2.1.5.3]tridecan-13-one,
12-ethyl-8-(2-phenoxyethyl)-4-oxa-8,12-diazadispiro[2.1.5.3]tridecan-13-one,
8-butyl-12-ethyl-4-oxa-8,12-diazadispiro[2.1.5.3]tridecan-13-one,
8-(2-ethoxyethyl)-12-ethyl-4-oxa-8,12-diazadispiro[2.1.5.3]tridecan-13-one,
8-cyclopentyl-12-ethyl-4-oxa-8,12-diazadispiro[2.1.5.3]tridecan-13-one,
4-ethyl-9-isopentyl-2,2-dimethyl-1-oxa-4,9-diazaspiro[5.5]undecan-3-one,
12-ethyl-8-(4-fluorobenzyl)-4-oxa-8,12-diazadispiro[2.1.5.3]tridecan-13-one,
8-benzyl-12-ethyl-4-oxa-8,12-diazadispiro[2.1.5.3]tridecan-13-one,
8-benzyl-12-methyl-4-oxa-8,12-diazadispiro[2.1.5.3]tridecan-13-one,
(R)-9-benzyl-4-ethyl-2-methyl-1-oxa-4,9-diazaspiro[5.5]undecan-3-one,
8-benzyl-12-isopropyl-4-oxa-8,12-diazadispiro[2.1.5.3]tridecan-13-one,
8,12-diethyl-4-oxa-8,12-diazadispiro[2.1.5.3]tridecan-13-one,
(S)-9-benzyl-4-ethyl-2-methyl-1-oxa-4,9-diazaspiro[5.5]undecan-3-one,
12-ethyl-8-(3-fluorobenzyl)-4-oxa-8,12-diazadispiro[2.1.5.3]tridecan-13-one,
8-(3,4-difluorobenzyl)-12-ethyl-4-oxa-8,12-diazadispiro[2.1.5.3]tridecan-13-one,
12-ethyl-8-(pyridin-3-ylmethyl)-4-oxa-8,12-diazadispiro[2.1.5.3]tridecan-13-one,
8-benzyl-12-propyl-4-oxa-8,12-diazadispiro[2.1.5.3]tridecan-13-one,
8-benzyl-12-isobutyl-4-oxa-8,12-diazadispiro[2.1.5.3]tridecan-13-one,
12-ethyl-8-(pyridin-4-ylmethyl)-4-oxa-8,12-diazadispiro[2.1.5.3]tridecan-13-one,
12-ethyl-8-(pyridin-2-ylmethyl)-4-oxa-8,12-diazadispiro[2.1.5.3]tridecan-13-one,
12-ethyl-8-propyl-4-oxa-8,12-diazadispiro[2.1.5.3]tridecan-13-one,
12-ethyl-8-(2-fluorobenzyl)-4-oxa-8,12-diazadispiro[2.1.5.3]tridecan-13-one,
9-benzyl-4-ethyl-2,2-dimethyl-1-oxa-4,9-diazaspiro[5.5]undecan-3-one,
8-cyclobutyl-12-ethyl-4-oxa-8,12-diazadispiro[2.1.5.3]tridecan-13-one,
8-cyclohexyl-12-ethyl-4-oxa-8,12-diazadispiro[2.1.5.3]tridecan-13-one,
12-ethyl-8-(tetrahydro-2H-pyran-4-yl)-4-oxa-8,12-diazadispiro[2.1.5.3]tridecan-13-one,
8-(2-(3-fluoropyridin-2-yl)ethyl)-12-isopropyl-4-oxa-8,12-diazadispiro[2.1.5.3]tridecane,
9-(2-(3-fluoropyridin-2-yl)ethyl)-4-isopropyl-1-oxa-4,9-diazaspiro[5.5]undecane,
(S)-9-(2-(3-fluoropyridin-2-yl)ethyl)-4-isopropyl-2-methyl-1-oxa-4,9-diazaspiro[5.5]undecane,
(R)-9-(2-(3-fluoropyridin-2-yl)ethyl)-4-isopropyl-2-methyl-1-oxa-4,9-diazaspiro[5.5]undecane,
8-(2-(3-fluoropyridin-2-yl)ethyl)-12-propyl-4-oxa-8,12-diazadispiro[2.1.5.3]tridecane,
12-ethyl-8-(2-(3-fluoropyridin-2-yl)ethyl)-4-oxa-8,12-diazadispiro[2.1.5.3]tridecane,
8-(2-(3-fluoropyridin-2-yl)ethyl)-12-isobutyl-4-oxa-8,12-diazadispiro[2.1.5.3]tridecane,
8-(2-(3-fluoropyridin-2-yl)ethyl)-12-methyl-4-oxa-8,12-diazadispiro[2.1.5.3]tridecane,
(S)-9-isopentyl-4-isopropyl-2-methyl-1-oxa-4,9-diazaspiro[5.5]undecan-3-one,
9-isopentyl-4-isopropyl-2,2-dimethyl-1-oxa-4,9-diazaspiro[5.5]undecan-3-one,
8-(2,5-difluorophenethyl)-12-isobutyl-4-oxa-8,12-diazadispiro[2.1.5.3]tridecane,
12-isobutyl-8-(2-(6-methoxypyridin-2-yl)ethyl)-4-oxa-8,12-diazadispiro[2.1.5.3]tridecane,
9-(3,3-dimethylbutyl)-4-propyl-1-oxa-4,9-diazaspiro[5.5]undecan-3-one,
9-isopentyl-4-propyl-1-oxa-4,9-diazaspiro[5.5]undecan-3-one,
9-(3,3-dimethylbutyl)-4-isopropyl-1-oxa-4,9-diazaspiro[5.5]undecan-3-one,
9-(3,3-dimethylbutyl)-4-ethyl-1-oxa-4,9-diazaspiro[5.5]undecan-3-one,
(S)-9-(3,3-dimethylbutyl)-4-ethyl-2-methyl-1-oxa-4,9-diazaspiro[5.5]undecan-3-one,
9-isopentyl-4-isopropyl-1-oxa-4,9-diazaspiro[5.5]undecan-3-one,
9-isopentyl-2,2-dimethyl-4-propyl-1-oxa-4,9-diazaspiro[5.5]undecan-3-one,
(S)-9-isopentyl-2-methyl-4-propyl-1-oxa-4,9-diazaspiro[5.5]undecan-3-one,
4-cyclopropyl-9-isopentyl-1-oxa-4,9-diazaspiro[5.5]undecan-3-one,
(S)-9-(2-isopropoxyethyl)-4-isopropyl-2-methyl-1-oxa-4,9-diazaspiro[5.5]undecan-3-one,
(S)-4-cyclopropyl-9-isopentyl-2-methyl-1-oxa-4,9-diazaspiro[5.5]undecan-3-one,
4-cyclopropyl-9-(2-isopropoxyethyl)-1-oxa-4,9-diazaspiro[5.5]undecan-3-one,
(S)-4-ethyl-9-(2-isopropoxyethyl)-2-methyl-1-oxa-4,9-diazaspiro[5.5]undecan-3-one,
9-(2-isopropoxyethyl)-4-isopropyl-2-methyl-1-oxa-4,9-diazaspiro[5.5]undecan-3-one,
(S)-4-cyclopropyl-9-(2-isopropoxyethyl)-2-methyl-1-oxa-4,9-diazaspiro[5.5]undecan-3-one,
(S)-9-(2-cyclopropylethyl)-4-ethyl-2-methyl-1-oxa-4,9-diazaspiro[5.5]undecan-3-one,
9-(2-cyclopropylethyl)-4-ethyl-1-oxa-4,9-diazaspiro[5.5]undecan-3-one,
(S)-9-(2-cyclopropylethyl)-4-isopropyl-2-methyl-1-oxa-4,9-diazaspiro[5.5]undecan-3-one,
(S)-9-(2-isopropoxyethyl)-2-methyl-4-propyl-1-oxa-4,9-diazaspiro[5.5]undecan-3-one,
8-(4-fluorobenzyl)-12-methyl-4-oxa-8,12-diazadispiro[2.1.5.3]tridecan-13-one,
4-ethyl-9-(4-fluorobenzyl)-2,2-dimethyl-1-oxa-4,9-diazaspiro[5.5]undecan-3-one,
(S)-9-(4-fluorobenzyl)-4-isopropyl-2-methyl-1-oxa-4,9-diazaspiro[5.5]undecan-3-one,
(R)-4-ethyl-9-(4-fluorobenzyl)-2-methyl-1-oxa-4,9-diazaspiro[5.5]undecan-3-one,
(S)-4-ethyl-9-(4-fluorobenzyl)-2-methyl-1-oxa-4,9-diazaspiro[5.5]undecan-3-one,
(S)-9-(4-chlorobenzyl)-4-ethyl-2-methyl-1-oxa-4,9-diazaspiro[5.5]undecan-3-one,
(R)-9-(4-chlorobenzyl)-4-ethyl-2-methyl-1-oxa-4,9-diazaspiro[5.5]undecan-3-one,
(R)-4-((4-ethyl-2-methyl-3-oxo-1-oxa-4,9-diazaspiro[5.5]undecan-9-yl)methyl)benzonitrile,
(R)-4-ethyl-9-(4-methoxybenzyl)-2-methyl-1-oxa-4,9-diazaspiro[5.5]undecan-3-one,
9-(4-chlorobenzyl)-4-ethyl-2,2-dimethyl-1-oxa-4,9-diazaspiro[5.5]undecan-3-one,
4-((4-ethyl-2,2-dimethyl-3-oxo-1-oxa-4,9-diazaspiro[5.5]undecan-9-yl)methyl)benzonitrile,
4-ethyl-9-(4-methoxybenzyl)-2,2-dimethyl-1-oxa-4,9-diazaspiro[5.5]undecan-3-one,
(S)-4-ethyl-2-methyl-9-neopentyl-1-oxa-4,9-diazaspiro[5.5]undecan-3-one,
(S)-4-ethyl-9-(4-methoxybenzyl)-2-methyl-1-oxa-4,9-diazaspiro[5.5]undecan-3-one,
(S)-4-((4-ethyl-2-methyl-3-oxo-1-oxa-4,9-diazaspiro[5.5]undecan-9-yl)methyl)benzonitrile,
(R)-4-((2,4-dimethyl-3-oxo-1-oxa-4,9-diazaspiro[5.5]undecan-9-yl)methyl)benzonitrile,
(S)-4-((4-isopropyl-2-methyl-3-oxo-1-oxa-4,9-diazaspiro[5.5]undecan-9-yl)methyl)benzonitrile,
(R)-9-(4-fluorobenzyl)-2,4-dimethyl-1-oxa-4,9-diazaspiro[5.5]undecan-3-one,
(S)-4-cyclopropyl-9-(4-fluorobenzyl)-2-methyl-1-oxa-4,9-diazaspiro[5.5]undecan-3-one,
(S)-9-benzyl-4-cyclopropyl-2-methyl-1-oxa-4,9-diazaspiro[5.5]undecan-3-one,
(S)-4-((4-cyclopropyl-2-methyl-3-oxo-1-oxa-4,9-diazaspiro[5.5]undecan-9-yl)methyl)benzonitrile,
(S)-4-((2,4-dimethyl-3-oxo-1-oxa-4,9-diazaspiro[5.5]undecan-9-yl)methyl)benzonitrile,
(S)-9-(4-fluorobenzyl)-2,4-dimethyl-1-oxa-4,9-diazaspiro[5.5]undecan-3-one,
(S)-9-benzyl-2,4-dimethyl-1-oxa-4,9-diazaspiro[5.5]undecan-3-one,
(S)-2,4-dimethyl-9-(4-(trifluoromethoxy)benzyl)-1-oxa-4,9-diazaspiro[5.5]undecan-3-one,
(S)-3-((4-ethyl-2-methyl-3-oxo-1-oxa-4,9-diazaspiro[5.5]undecan-9-yl)methyl)benzonitrile,
(S)-4-((4-ethyl-2-methyl-3-oxo-1-oxa-4,9-diazaspiro[5.5]undecan-9-yl)methyl)-2-fluorobenzonitrile,
(S)-5-((4-ethyl-2-methyl-3-oxo-1-oxa-4,9-diazaspiro[5.5]undecan-9-yl)methyl)-2-fluorobenzonitrile,
(S)-9-(2,4-difluorobenzyl)-4-ethyl-2-methyl-1-oxa-4,9-diazaspiro[5.5]undecan-3-one,
(S)-3-((4-isopropyl-2-methyl-3-oxo-1-oxa-4,9-diazaspiro[5.5]undecan-9-yl)methyl)benzonitrile,
(S)-9-benzyl-4-isopropyl-2-methyl-1-oxa-4,9-diazaspiro[5.5]undecan-3-one,
(R)-9-benzyl-4-isopropyl-2-methyl-1-oxa-4,9-diazaspiro[5.5]undecan-3-one,
(R)-9-(4-fluorobenzyl)-4-isopropyl-2-methyl-1-oxa-4,9-diazaspiro[5.5]undecan-3-one,
(R)-4-((4-isopropyl-2-methyl-3-oxo-1-oxa-4,9-diazaspiro[5.5]undecan-9-yl)methyl)benzonitrile,
8-(2-(3-chloropyridin-2-yl)ethyl)-12-isobutyl-4-oxa-8,12-diazadispiro[2.1.5.3]tridecane,
2-(2-(12-isobutyl-4-oxa-8,12-diazadispiro[2.1.5.3]tridecan-8-yl)ethyl)nicotinonitrile,
8-(2-(3-fluoropyridin-2-yl)ethyl)-12-isopentyl-4-oxa-8,12-diazadispiro[2.1.5.3]tridecane,
8-(2-(3-fluoropyridin-2-yl)ethyl)-12-neopentyl-4-oxa-8,12-diazadispiro[2.1.5.3]tridecane,
12-(sec-butyl)-8-(2-(3-fluoropyridin-2-yl)ethyl)-4-oxa-8,12-diazadispiro[2.1.5.3]tridecane,
4-ethyl-9-(2-(3-fluoropyridin-2-yl)ethyl)-2,2-dimethyl-1-oxa-4,9-diazaspiro[5.5]undecane,
(S)-4-ethyl-9-(2-(3-fluoropyridin-2-yl)ethyl)-2-methyl-1-oxa-4,9-diazaspiro[5.5]undecane,
(S)-9-(2-(3-fluoropyridin-2-yl)ethyl)-2-methyl-4-propyl-1-oxa-4,9-diazaspiro[5.5]undecane,
4-Isopropyl-2-methyl-9-phenethyl-1-oxa-4,9-diazaspiro[5.5]undecane,
4-(2-cyclopropylethyl)-2-methyl-9-phenethyl-1-oxa-4,9-diazaspiro[5.5]undecane,
4-(cyclopropylmethyl)-9-phenethyl-1-oxa-4,9-diazaspiro[5.5]undecane,
4-Ethyl-2-methyl-9-phenethyl-1-oxa-4,9-diazaspiro[5.5]undecane, and
4-isopropyl-2,2-dimethyl-9-phenethyl-1-oxa-4,9-diazaspiro[5.5]undecane.

35. The method according to claim 16, wherein the compound of Formula (I) is selected from the group consisting of:
(S)-9-(4-fluorobenzyl)-2,4-dimethyl-1-oxa-4,9-diazaspiro[5.5]undecan-3-one and
(S)-4-((4-ethyl-2-methyl-3-oxo-1-oxa-4,9-diazaspiro[5.5]undecan-9-yl)methyl)-2-fluorobenzonitrile.

36. The method according to claim 16, wherein providing neuroprotection and/or treating motoneuron degeneration comprises one or more of the following: treating ALS, preventing motoneuron death, enhancing motoneuron survival, slowing motoneuron degeneration, preserving neuromuscular junctions, protecting spinal motoneurons and reducing astroglial activation.

37. The method according to claim 16, wherein the compound of Formula (I) is administered at a dose of 0.05 to 100 mg/kg/day.

38. The method according to claim 37, wherein the compound of Formula (I) is administered at a dose of 0.05 to 50 mg/kg/day.

39. The method according to claim 37, wherein the compound of Formula (I) is administered at a dose of 0.05 to 25 mg/kg/day.

40. The method according to claim 37, wherein the compound of Formula (I) is administered at a dose of 0.05 to 10 mg/kg/day.

41. The method according to claim 37, wherein the compound of Formula (I) is administered at a dose of 0.05 to 5 mg/kg/day.

42. The method according to claim 37, wherein the compound of Formula (I) is administered at a dose of 0.5 to 5 mg/kg/day.

43. The method according to claim 16, wherein the compound of Formula (I) is administered in the form of a pharmaceutical composition comprising the compound of Formula (I), or a pharmaceutically acceptable salt thereof, and a pharmaceutically acceptable carrier, adjuvant or vehicle.
